(19) United States
(12) Patent Application Publication
(10) Pub. No.: US 2021/0147905 A1
(43) Pub. Date: May 20, 2021

(54) FLEXIBLE DETECTION SYSTEMS

(71) Applicant: Akoya Biosciences, Inc., Menlo Park, CA (US)

(72) Inventors: Julia Kennedy-Darling, Redwood City, CA (US); Joseph Kim, Menlo Park, CA (US); Gajalakshmi Dakshinamoorthy, Redwood City, CA (US); Brian McKelligon, Portland, OR (US)

(21) Appl. No.: 17/161,267

(22) Filed: Jan. 28, 2021

Related U.S. Application Data
(63) Continuation of application No. PCT/US2020/012813, filed on Jan. 8, 2020.
(60) Provisional application No. 62/789,935, filed on Jan. 8, 2019.

(51) Int. Cl. C12Q 1/6804

(52) U.S. Cl. CPC C12Q 1/6804; G01N 2458/10

(57) ABSTRACT

Provided herein are methods, kits, and compositions for the detection of an element of a biological sample using an antibody conjugated to a first oligonucleotide, which connects to a labelled oligonucleotide for detection via an oligonucleotide partially complementary to the first oligonucleotide and partially complementary to the second oligonucleotide.

### CROSS-REFERENCE

This application is a continuation of PCT Application No. PCT/US2020/012813, filed Jan. 8, 2020; which claims priority to U.S. Provisional Patent Application No. 62/789,935, filed Jan. 8, 2019; which are entirely incorporated herein by reference.

### BACKGROUND OF THE INVENTION

Antibodies were first employed in tissue section analysis in 1942 to visualize pneumococcal antigens in organ biopsies from mice infused with live bacteria. Since that time, immunohistochemistry has become a mainstay of clinical diagnostics and basic research.

### SUMMARY OF THE INVENTION

Provided herein are methods comprising: contacting a biological sample with an antibody or antibody fragment that is conjugated to a first oligonucleotide; contacting said first oligonucleotide with a first binding region of a second oligonucleotide; contacting a second binding region of said second oligonucleotide with a third oligonucleotide, wherein said third oligonucleotide comprises a detection component; thereby connectively coupling said biological sample to said detection component. In some embodiments, said biological sample comprises at least one component selected from the group consisting of: cultured cells, biological tissue, biological fluid, a homogenate, and an unknown biological sample. In some embodiments, said biological sample comprises material that is selected from the group consisting of: human origin, mouse origin, rat origin, cow origin, pig origin, sheep origin, rabbit origin, monkey origin, fruit fly origin, frog origin, nematode origin, fish origin, hamster origin, guinea pig origin, and woodchuck origin. In some embodiments, said biological sample comprises material that is selected from the group consisting of: animal origin, plant origin, bacteria origin, fungus origin, and protist origin. In some embodiments, said biological sample comprises a component selected from the group consisting of: virus, viral vector, and prion. In some embodiments, said biological sample is fresh, frozen, or fixed. In some embodiments, said biological sample is immobilized on a surface. In some embodiments, said surface is a slide, a plate, a well, a tube, a membrane, a film, or a bead. In some embodiments, said biological sample is immobilized within a three-dimensional structure. In some embodiments, said three-dimensional structure is a frozen tissue, a paraffin block, or a frozen liquid. In some embodiments, said antibody or antibody fragment comprises an IgG, an IgM, a monoclonal antibody, a scFv, a nanobody, a Fab, or a diabody. In some embodiments, said antibody or antibody fragment is specific for an element of the sample. In some embodiments, said element of the sample is frozen-fixed sample, a protein, a DNA molecule, an RNA molecule, or a lipid. In some embodiments, said first oligonucleotide comprises a plurality of ribonucleic acids. In some embodiments, said first oligonucleotide comprises a plurality of deoxyribonucleic acids. In some embodiments, said first oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said first oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said first oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said first oligonucleotide comprises one or more synthetic nucleotides. In some embodiments, said first oligonucleotide is wholly single stranded. In some embodiments, said first oligonucleotide is partially double stranded. In some embodiments, said first binding region of said second oligonucleotide is complimentary to at least a portion of said first oligonucleotide. In some embodiments, said first binding region of the second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said first binding region of the second oligonucleotide is between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said first binding region of the second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said first binding region of said second oligonucleotide comprises one or more synthetic nucleotides. In some embodiments, said second oligonucleotide comprises a plurality of ribonucleic acids. In some embodiments, said second oligonucleotide comprises a plurality of deoxyribonucleic acids. In some embodiments, said second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said second oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said second oligonucleotide comprises one or more synthetic nucleotides. In some embodiments, said second oligonucleotide is wholly single stranded. In some embodiments, said second oligonucleotide is partially double stranded. In some embodiments, said second binding region of said second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said second binding region of said second oligonucleotide is between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said second binding region of said second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said second binding region of said second oligonucleotide comprises one or more synthetic nucleotides. In some embodiments, said second binding region of said second oligonucleotide is complimentary to at least a portion of said third oligonucleotide. In some embodiments, said third oligonucleotide comprises a plurality of ribonucleic acids. In some embodiments, said third oligonucleotide comprises a plurality of deoxyribonucleic acids. In some embodiments, said third oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said third oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said third oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said third binding region of said second oligonucleotide comprises one or more synthetic nucleotides. In some embodiments, said third oligonucleotide is wholly single stranded. In some embodiments, said third oligonucleotide is partially double stranded. In some embodiments, said third oligonucleotide is partially complimentary to said second binding region of said second oligonucleotide. In some embodiments, said third oligonucleotide is fully complimentary to said second binding region of said second oligonucleotide. In some embodiments, said detection component comprises a fluorophore, a radioactive isotope, or a compound capable of producing a colorimetric reaction. In some embodiments, said detection component is located at the 3′ end of said third oligonucleotide. In some embodiments, said detection component is located at the 5′ end of said third oligonucleotide. In some embodiments, said detection component is located between the 3′ end and the 5′ end of said third oligonucleotide. In some embodiments, said detection component be removed. Some embodiments further comprise the step of immobilizing said biological sample on a surface prior to contacting said sample with said antibody or antibody fragment. Some embodiments further comprise detection of the detection component after contacting said second binding region of said second oligonucleotide with said third oligonucleotide. In some embodiments, the method is performed in a stepwise fashion. In some embodiments, one or more steps are performed simultaneously. In some embodiments, laser capture microdissection is performed after contacting said second binding region of said second oligonucleotide with said third oligonucleotide.

Also provided herein are kits comprising: an antibody or antibody fragment that is conjugated to a first oligonucleotide; a second oligonucleotide comprising a first binding region and a second binding region, wherein the first binding region of said second oligonucleotide is complimentary to at least a portion of said first oligonucleotide; and a third oligonucleotide comprising a detection component, wherein said second binding region of said second oligonucleotide is complimentary to at least a portion of said third oligonucleotide. In some embodiments, said antibody or antibody fragment comprises an IgG, an IgM, a monoclonal antibody, a scFv, a nanobody, a Fab, or a diabody. In some embodiments, a non-specific bound antibody comprises less than 1%, less than 2%, less than 3%, less than 4%, less than 5%, less than 10%, less than 15%, less than 20%, less than 25%, less than 30%, less than 35%, or less than 40% of said antibody bound to the sample. In some embodiments, said first oligonucleotide comprises a plurality of ribonucleic acids. In some embodiments, said first oligonucleotide comprises a plurality of deoxyribonucleic acids. In some embodiments, said first oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said first oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said first oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said first oligonucleotide comprises one or more synthetic nucleotides. In some embodiments, said first oligonucleotide is wholly single stranded. In some embodiments, said first oligonucleotide is partially double stranded. In some embodiments, said first binding region of said second oligonucleotide is complimentary to at least a portion of said first oligonucleotide. In some embodiments, said first binding region of the second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said first binding region of the second oligonucleotide is between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said first binding region of the second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said first binding region of said second oligonucleotide comprises one or more synthetic nucleotides. In some embodiments, said second oligonucleotide comprises a plurality of ribonucleic acids. In some embodiments, said second oligonucleotide comprises a plurality of deoxyribonucleic acids. In some embodiments, said second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said second oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said second oligonucleotide comprises one or more synthetic nucleotides. In some embodiments, said second oligonucleotide is wholly single stranded. In some embodiments, said second oligonucleotide is partially double stranded. In some embodiments, said second binding region of said second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said second binding region of said second oligonucleotide is between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said second binding region of said second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said second binding region of said second oligonucleotide comprises one or more synthetic nucleotides. In some embodiments, said second binding region of said second oligonucleotide is complimentary to at least a portion of said third oligonucleotide. In some embodiments, said third oligonucleotide comprises a plurality of ribonucleic acids. In some embodiments, said third oligonucleotide comprises a plurality of deoxyribonucleic acids. In some embodiments, said third oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said third oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said third oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said third binding region of said second oligonucleotide comprises one or more synthetic nucleotides. In some embodiments, said third oligonucleotide is wholly single stranded. In some embodiments, said third oligonucleotide is partially double stranded. In some embodiments, said third oligonucleotide is partially complimentary to said second binding region of said second oligonucleotide. In some embodiments, said third oligonucleotide is fully complimentary to said second binding region of said second oligonucleotide. In some embodiments, said detection component comprises a fluorophore, a radioactive isotope, or a compound capable of producing a colorimetric reaction. In some embodiments, said detection component is located at the 3′ end of said third oligonucleotide. In some embodiments, said detection component is located at the 5′ end of said third oligonucleotide. In some embodiments, said detection component is located between the 3′ end and the 5′ end of said third oligonucleotide. In some embodiments, said detection component can be removed.

Also provided herein are compositions comprising: an antibody or antibody fragment that is conjugated to a first oligonucleotide; wherein said first oligonucleotide is connected via base pairs to a first binding region of a second oligonucleotide; wherein a second binding region of said second oligonucleotide is connected via base pairs to a third oligonucleotide; and wherein said third oligonucleotide comprises a detection component. In some embodiments, said antibody or antibody fragment comprises an IgG, an IgM, a monoclonal antibody, a scFv, a nanobody, a Fab, or a diabody. In some embodiments, a non-specific bound antibody comprises less than 1%, less than 2%, less than 3%, less than 4%, less than 5%, less than 10%, less than 15%, less than 20%, less than 25%, less than 30%, less than 35%, or less than 40% of said antibody bound to the sample. In some embodiments, said first oligonucleotide comprises a plurality of ribonucleic acids. In some embodiments, said first oligonucleotide comprises a plurality of deoxyribonucleic acids. In some embodiments, said first oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said first oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said first oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said first oligonucleotide comprises one or more synthetic nucleotides. In some embodiments, said first oligonucleotide is wholly single stranded. In some embodiments, said first oligonucleotide is partially double stranded. In some embodiments, said first binding region of said second oligonucleotide is complimentary to at least a portion of said first oligonucleotide. In some embodiments, said first binding region of the second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said first binding region of the second oligonucleotide is between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said first binding region of the second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said first binding region of said second oligonucleotide comprises one or more synthetic nucleotides. In some embodiments, said second oligonucleotide comprises a plurality of ribonucleic acids. In some embodiments, said second oligonucleotide comprises a plurality of deoxyribonucleic acids. In some embodiments, said second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said second oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said second oligonucleotide comprises one or more synthetic nucleotides. In some embodiments, said second oligonucleotide is wholly single stranded. In some embodiments, said second oligonucleotide is partially double stranded. In some embodiments, said second binding region of said second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said second binding region of said second oligonucleotide is between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said second binding region of said second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said second binding region of said second oligonucleotide comprises one or more synthetic nucleotides.

The method of claim 1, wherein said second binding region of said second oligonucleotide is complimentary to at least a portion of said third oligonucleotide. In some embodiments, said third oligonucleotide comprises a plurality of ribonucleic acids. In some embodiments, said third oligonucleotide comprises a plurality of deoxyribonucleic acids. In some embodiments, said third oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some embodiments, said third oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some embodiments, said third oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some embodiments, said third binding region of said second oligonucleotide comprises one or more synthetic nucleotides. In some embodiments, said third oligonucleotide is wholly single stranded. In some embodiments, said third oligonucleotide is partially double stranded. In some embodiments, said third oligonucleotide is partially complimentary to said second binding region of said second oligonucleotide. In some embodiments, said third oligonucleotide is fully complimentary to said second binding region of said second oligonucleotide. In some embodiments, said detection component comprises a fluorophore, a radioactive isotope, or a compound capable of producing a colorimetric reaction. In some embodiments, said detection component is located at the 3′ end of said third oligonucleotide. In some embodiments, said detection component is located at the 5′ end of said third oligonucleotide. In some embodiments, said detection component is located between the 3′ end and the 5′ end of said third oligonucleotide. In some embodiments, said detection component be removed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a schematic representing some embodiments of compositions herein. 1 represents an antibody or antibody fragment. 2 represents a first oligonucleotide. 3 represents a second oligonucleotide. 4 represents a third oligonucleotide. 5 represents a detection component.

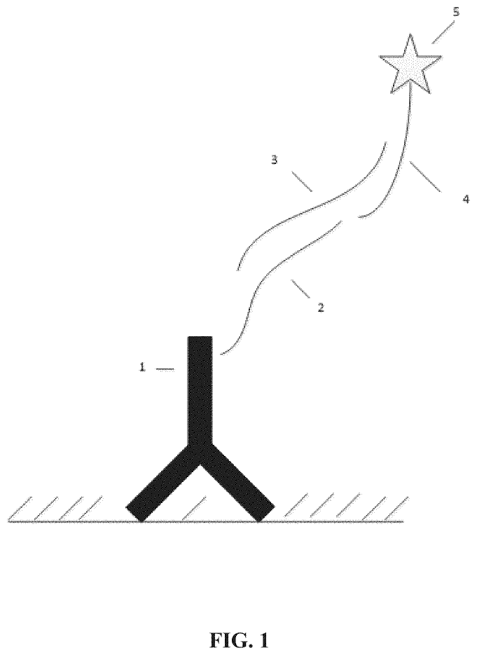

FIG. 2 illustrates examples of how compositions described herein might appear. Variations can include but are not limited to: non-binding regions, overhangs, loops, or additional oligonucleotides. A represents an example where the second oligonucleotide connects the first oligonucleotide with the labeled third oligonucleotide; B represents an example where the third oligonucleotide is entirely complimentary to the second oligonucleotide; C represents an example wherein the first binding region comprises a section which is not complimentary to the first oligonucleotide; D represents an example wherein the first oligonucleotide can have a secondary structure; E represents an example wherein the first binding region does not extend to the edge of the second oligonucleotide; F represents an example wherein the segment of the third oligonucleotide which binds to the second binding region comprises a section which is not complimentary to the second binding region; G represents an example wherein the third oligonucleotide can have a secondary structure; H represents an example wherein the first binding region is not complimentary to the end of the first oligonucleotide; I represents an example wherein the second oligonucleotide is circular; J represents an example wherein the segment of the third oligonucleotide which binds to the second binding region comprises a segment which is not complimentary to the second binding region and has a secondary structure; K represents an example wherein the second binding region does not extend to the end of the second oligonucleotide; L represents an example wherein the third oligonucleotide has a secondary structure; M represents an example wherein neither the first binding region nor the second binding region extends to either edge of the second oligonucleotide; N represents an example wherein the second oligonucleotide can be a partially double stranded oligonucleotide; O represents an example wherein the third oligonucleotide can be partially double stranded; P represents an example wherein the second oligonucleotide can comprise three partially complimentary oligonucleotides as a partially double stranded oligonucleotide; Q represents an example wherein the second binding region is interrupted by a region which is not complimentary to the third oligonucleotide, while the segment of the third oligonucleotide which is complimentary to the second binding region is interrupted by a region which is not complimentary to the second oligonucleotide.

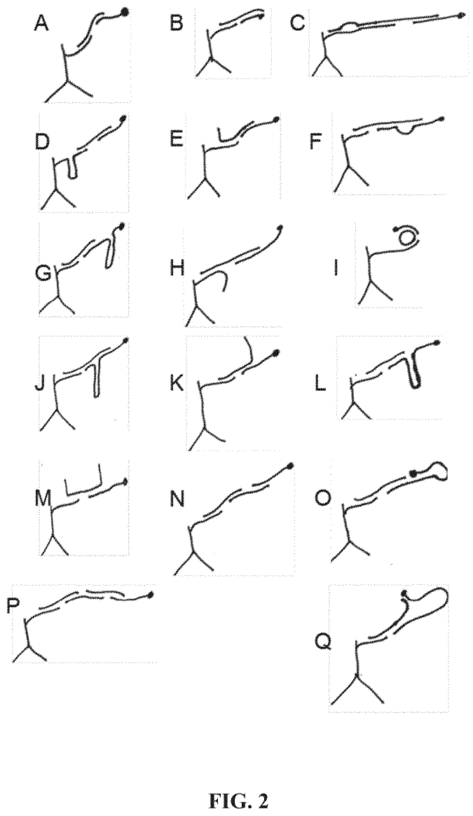

FIG. 3 illustrates activation of the detection component in the case the detection component is a fluorophore by (i) single photon excitation, (ii) double photon excitation, and (iii) triple photon excitation.

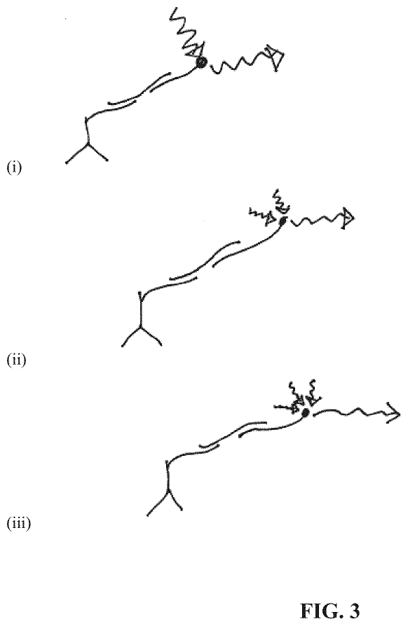

FIG. 4 illustrates possible configurations of a detection component, which can include a detection component located at either end of a third oligonucleotide ((i) and (ii)), in the middle of a third oligonucleotide (iii), multiple detection components on a third oligonucleotide (iv), and a FRET detection system ((v) and (vi)).

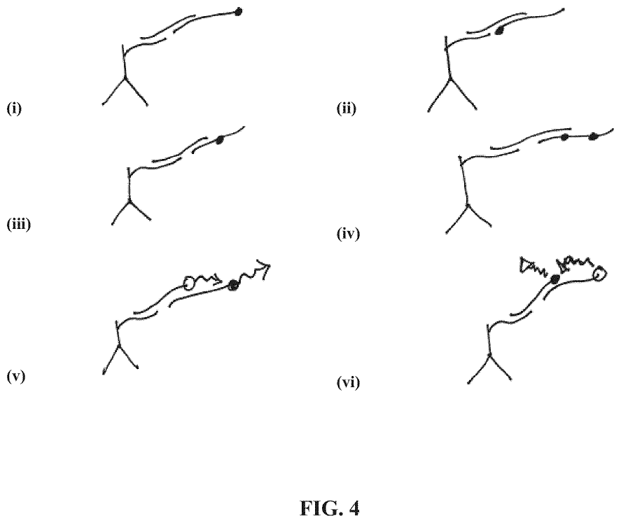

### DETAILED DESCRIPTION OF THE INVENTION

Described herein are methods, kits, and compositions for the identification of an element of a biological sample. Briefly, a biological sample can be in contact with an antibody or antibody fragment conjugated to a first oligonucleotide, such that at least a portion of the antibody or antibody fragment is in contact with an element of the biological sample. The antibody or antibody fragment can be conjugated with a first oligonucleotide, which can have a first binding region. The first oligonucleotide can then contact a second oligonucleotide. In some cases, the first binding region of the first oligonucleotide can contact a first binding region of a second oligonucleotide. A second binding region of the second oligonucleotide can then contact a third oligonucleotide, wherein the third oligonucleotide can comprise a detection component. Thus, the element of the biological sample can be linked to the detection component via an antibody or antibody fragment and a plurality of oligonucleotides. The detection component can then be detected to determine qualitatively or quantitatively the presence of the element of the biological sample.

### Samples

A sample can be a biological sample. A sample can be fresh, frozen, or fixed (e.g., chemically fixed). A sample can be of animal, plant, bacteria, fungus, or protist origin. In some cases, a sample can be that of a human, mouse, rat, cow, pig, sheep, monkey, rabbit, fruit fly, frog, nematode or woodchuck. A sample can comprise cells (e.g., isolated cells, immortalized cells, primary cells, cultured cells, or cells of a tissue or organism), biological tissue, biological fluid, a homogenate, or it can be an unknown sample. In some cases, a sample can comprise a pathogen. The pathogen can be cultured or uncultured. A pathogen can be an infection of a sample. In some cases, a pathogen can be an infection of a cell, fluid, tissue, organ, or microbiome of an organism a sample is collected from. In some cases, a sample can comprise a pathogen which is a yeast cell, a bacterial cell, a virus, a viral vector or a prion.

A sample can be a tissue section. In some cases, tissue section can refer to a piece of tissue that has been obtained from a subject, optionally fixed, sectioned, and mounted on a planar surface, e.g., a microscope slide.

A sample can be a planar sample. In some cases, a sample can be immobilized on a surface. In some cases, the surface can be a slide, a plate, a well, a tube, a membrane, a film, or a bead. In some cases, a sample can be contacting a slide. A sample contacting a slide can be attached to the slide such that the sample is effectively immobilized. This can be accomplished for example by fixation or by freezing the sample. Likewise, a sample can be immobilized on another type of surface using a same or similar attachment technique.

In some cases, a sample can be a formalin-fixed paraffin embedded (FFPE) tissue section. FFPE can refer to a piece of tissue, e.g., a biopsy that has been obtained from a subject, fixed, for example in formalin or formaldehyde (e.g., 3%-5% formalin or formaldehyde in phosphate buffered saline) or Bouin solution, embedded in wax, cut into thin sections, and then mounted on a microscope slide.

A sample can be a non-planar sample. A non-planar sample can be a sample that is not substantially flat, e.g., a whole or part organ mount (e.g., of a lymph node, brain, liver, etc.), that has been made transparent by means of a refractive index matching technique such as Clear Lipid-exchanged Acrylamide-hybridized Rigid Imaging-compatible Tissue-hydrogel (CLARITY). See, e.g., Roberts et al., J Vis Exp. 2016; (112): 54025. Clearing agents such as Benzyl-Alcohol/Benzyl Benzoate (BABB) or Benzyl-ether may be used to render a specimen transparent.

The sample may be fixed using an aldehyde, an alcohol, an oxidizing agent, a mercurial, a picrate, or HOPE fixative. In some instances, a sample can be fixed using acetone, formaldehyde, formalin, paraformaldehyde, ethanol, or methanol. The sample may alternatively be fixed using heat fixation. Fixation may be achieved via immersion or perfusion.

In some cases, the biological sample may be frozen. In some cases, the biological sample may be frozen at less than 0° C., less than −10° C., less than −20° C., less than −30° C., less than −40° C., less than −50° C., less than −60° C., less than −70° C., or less than −80° C.

In some cases, a biological sample can be immobilized in a three-dimensional form. Said three-dimensional form can be a frozen block, a paraffin block, or a frozen liquid. For example, a biological sample can be a block of frozen animal tissue in an optimal cutting temperature (OCT) compound. Such a block of tissue can be frozen or fixed. In some cases, a block of tissue can be cut to reveal a surface which can be the surface contacted by the antibody or antibody fragment. Sometimes, a block can be sliced such that serial surfaces of the block can be contacted by the antibody or antibody fragment. In such cases, data which is three-dimensional or approximates three-dimensional data can be acquired.

### Biological Features of Interest

A sample can comprise a biological feature of interest. A biological feature of interest can comprise any part of a sample which can be measured using methods described herein. In some cases, a biological feature of interest can comprise a part of a sample that can be indicated by binding to a capture agent. A biological feature of interest can be a control feature such as a housekeeping feature such as for normalization (e.g., actin), a feature which can identify a part of a cell (e.g., a protein associated with a nucleus, nuclear membrane, endoplasmic reticulum, mitochondria, cell membrane, or other part of the cell), a feature which can identify a type of cell (e.g., a cell surface marker or a protein expressed in a particular cell type, such as an immune cell or a cancer cell), or another feature of interest. In some cases, a biological feature of interest can be a marker of a disease, such as cancer, diabetes, a cardiac disease, a pulmonary disease, an autoimmune disease, an inflammatory disease, or another type of disease. In some cases, a biological feature of interest can be a marker of injury or a marker that is present during would healing. In some cases, a biological feature of interest can be a marker that can indicate a healthy cell. In some cases, a biological feature of interest can be a feature of interest for diagnostic, drug discovery, research, identification, or optimization purposes. In some cases, a biological feature of interest can be an antigen. In some cases, a biological feature of interest can comprise a cell wall, a nucleus, cytoplasm, a membrane, keratin, a muscle fiber, collagen, bone, a protein, a nucleic acid (e.g., mRNA or genomic DNA, etc), fat, etc. A biological feature of interest can also be indicated by immunohistological methods, e.g., using a capture agent that is linked to an oligonucleotide.

A sample can comprise a number of biological features of interest that can be detected using the methods herein. In some cases, the multiplexing features of the method herein (e.g., allowing label to be removed while keeping the capture agents intact on the sample, thus allowing for several or many iterations of the method on a single sample) can be used to detect many biological features of interest. In some cases, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 biological features of interest can be detected. In some cases, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, or about 100 biological features of interest can be detected. In some cases, more biological features can be detected in a sample using the present methods than by using other methods, such as non-multiplexed methods, methods wherein a capture agent must be stripped from the sample, methods not including fixing or crosslinking a capture agent to a sample, methods without amplification, or methods with an amplification method different than RCA.

A biological feature of interest can comprise a marker. A marker can be a molecule within a cell, such as a protein, that can inform on the type, disease status, pathogenicity, senescence, or other property of a cell. A marker can in some cases inform a type of cell, such as a lymph cell, a T-cell, a B-cell, a neutrophil, a macrophage, a germ cell, a stem cell, a neural cell, a cancer cell, a healthy cell, an aged cell, an infected cell, or a cell belonging to a particular organ (e.g., a cardiac cell, a Sertoli cell, a hepatocyte, a dermal cell, a thyroid cell, a lung cell, an intestinal cell, a tonsil cell, a muscle cell, a bone cell, a retinal cell such as a rod or a cone, or a cell of another organ). In some cases, a marker can be used to identify a pathogen.

A marker can be a disease marker. A disease marker can be a marker (e.g., a protein) that can be altered in shape, activity, quantity, location, or whether or not it is present or not in a cell having a given disease state. For example, a disease marker can comprise a cancer marker (e.g., a breast cancer marker, a pancreatic cancer marker, a lymphoma marker, a head and neck cancer marker, a gastric cancer marker, a testicular cancer marker, a leukemia marker, a hepatocellular cancer marker, a lung cancer marker, a melanoma marker, an ovarian cancer marker, a thyroid cancer marker, or a marker of another type of cancer), an infectious disease marker (e.g., a marker of a disease caused by a pathogen, such as a marker on the pathogen or a marker of a cell or tissue infected by the pathogen), or a genetic disease marker.

A marker can be a diagnostic marker. A diagnostic marker can be for example a specific biochemical in the body which has a particular molecular feature that makes it useful for detecting a disease, measuring the progress of disease or the effects of treatment, or for measuring a process of interest.

A marker can be a low-level marker, such as a low-level surface marker.

### Capture Agents

A capture agent can be a molecule which can bind to a sample. In some cases, a capture agent can bind to a biological feature of interest of a sample. In some cases, a capture agent can specifically bind to a complementary site on a biological feature in a sample. Briefly, a biological feature of interest can be a feature of a sample which can be detected using a capture agent using methods described herein. In some cases, a biological feature of interest can be bound by the capture agent.

A capture agent can be a molecule capable of binding a biological feature. In some cases, a capture agent can comprise a protein, a peptide, an aptamer, or an oligonucleotide. In some cases, a capture agent can comprise an antibody or antigen binding fragment thereof. In some instances, an antibody or an antigen-binding fragment thereof can comprise an isolated antibody or antigen-binding fragment thereof, a purified antibody or antigen-binding fragment thereof, a recombinant antibody or antigen-binding fragment thereof, a modified antibody or antigen-binding fragment thereof, or a synthetic antibody or antigen-binding fragment thereof. It would be understood that antibodies described herein can be modified as known in the art.

A capture agent that is an antibody or antigen binding fragment thereof can comprise a variable region. In some cases, the variable region can comprise a part of an antibody or antigen binding fragment thereof that can contact or specifically bind a sample to bind with a biological feature of interest. A variable region can refer to the variable region of an antibody light chain, the variable region of an antibody heavy chain, or a combination of the variable region of an antibody light chain and the variable region of an antibody light chain. In some cases, capture agents which bind different biological features of interest can comprise variable regions which are different in amino acid sequence, protein modifications, three-dimensional structure, or a combination thereof.

A capture agent comprising an antibody or antigen binding fragment thereof can comprise antibody or antibody fragment can comprise an IgG, an IgM, a polyclonal antibody, a monoclonal antibody, a scFv, a nanobody, a Fab, or a diabody. In some cases, an antibody or antigen binding fragment thereof can be of mouse, rat, rabbit, human, camelid, or goat origin. In some cases, an antibody or antigen binding fragment thereof can be raised against a human, mouse, rat, cow, pig, sheep, monkey, rabbit, fruit fly, frog, nematode or woodchuck antigen. In some cases, an antibody or antigen binding fragment thereof can be raised against an animal, plant, bacteria, fungus, or protist antigen. In some cases, the antibody or antigen binding fragment thereof can be raised against a virus, a viral vector, or a prion.

In some cases, the method may comprise labeling the sample with the plurality of capture agents. This step involves contacting the sample (e.g., an FFPE section mounted on a planar surface such as a microscope slide) with all of the capture agents, en masse under conditions by which the capture agents can bind to biological features of interest in the sample. Methods for binding antibodies and aptamers to sites in the sample can be well known.

A capture agent can be in a buffer. In some cases, a capture agent can be applied to a sample in a buffer. A buffer comprising a capture agent can comprise properties which can allow the capture agent to be configured or folded in a state in which the capture agent can bind to a biological feature of interest. In some cases, a buffer comprising a capture agent can comprise properties which can promote binding of the capture agent to a biological feature of interest. In some cases, a buffer comprising a capture agent can comprise properties which can be non-destructive to the capture agent, non-destructive to an oligonucleotide, non-destructive to the sample, or non-destructive to the biological feature of interest.

A capture agent can have specificity for a biological feature of interest. In some cases, a capture agent can have specificity for only one biological feature of interest. In some cases, a capture agent can have specificity for a biological feature of interest that is greater than the specificity of that capture agent for a different biological feature of interest. In some cases, a capture agent can have a specificity for one biological feature of interest that is so much greater than its specificity for other biological features of interest that it can be used to reliably detect the first biological feature of interest.

A capture agent can have affinity for an element of the sample. In some cases, affinity can refer to how fast or how strong the antibody can bind to an element. Affinity can sometimes be described by the dissociation constant (Kd). A capture agent can have a Kd of no more than 10⁻⁴ M, no more than 10⁻⁵ M, no more than 10⁻⁶ M, no more than 10⁻⁷ M, no more than 10⁻⁸M, no more than 10⁻⁹M, no more than 10⁻¹⁰ M, no more than 10⁻¹¹M, no more than 10⁻¹² M, no more than 10⁻¹³ M, or no more than 10⁻¹⁴ M. In some cases, a capture agent can have a Kd of about 10⁻⁴ M, about 10⁻⁵ M, about 10⁻⁶ M, about 10⁻⁷ M, about 10⁻⁸ M, about 10⁻⁹ M, about 10⁻¹⁰ M, about 10⁻¹¹M, about 10⁻¹² M, about 10⁻¹³ M, or about 10⁻¹⁴ M.

A capture agent can bind to a biological feature of interest at a binding site on a biological feature of interest. Such a binding site, for example, can be an epitope. In some cases, an epitope can be a part of a biological feature of interest. In such a case, the biological feature of interest can comprise an antigen. In some cases, an epitope can bind a capture agent that is an antibody or antigen binding fragment thereof. In such cases, the variable region of the antibody or antigen binding fragment thereof can bind the biological feature of interest at its epitope.

In some cases, a capture agent can be applied to a sample in excess.

In some cases, after a capture agent is contacted with the sample, it can be allowed to incubate for an amount of time. In some cases, a capture agent can be incubated on a sample for at least 30 seconds, at least 1 minute, at least 2 minutes, at least 3 minutes, at least 4 minutes, at least 5 minutes, at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 1.5 hours, at least 2 hours, at least 2.5 hours, at least 3 hours, at least 4 hours, at least 5 hours, or at least 6 hours. In some cases, a capture agent can be incubated on a sample for no longer than 30 seconds, no longer than 1 minute, no longer than 2 minutes, no longer than 3 minutes, no longer than 4 minutes, no longer than 5 minutes, no longer than 10 minutes, no longer than 15 minutes, no longer than 20 minutes, no longer than 25 minutes, no longer than 30 minutes, no longer than 35 minutes, no longer than 40 minutes, no longer than 45 minutes, no longer than 50 minutes, no longer than 55 minutes, no longer than 60 minutes, no longer for 1.5 hours, no longer than 2 hours, no longer than 2.5 hours, no longer than 3 hours, no longer than 3.5 hours, no longer than 4 hours, no longer than 4.5 hours, no longer than 5 hours, no longer than 5.5 hours, or no longer than 6 hours. In some cases, a capture agent can be incubated on a sample for between 30 seconds and 6 hours, between 30 seconds and 3 hours, between 30 seconds and 60 minutes, between 30 seconds and 45 minutes, between 30 seconds and 30 minutes, between 30 seconds and 15 minutes, between 30 seconds and 5 minutes, between 30 seconds and 1 minute, between 1 minute and 6 hours, between 1 minute and 3 hours, between 1 minute and 60 minutes, between 1 minute and 45 minutes, between 1 minute and 30 minutes, between 1 minute and 15 minutes, between 1 minute and 5 minutes, between 5 minutes and 6 hours, between 5 minutes and 3 hours, between 5 minutes and 60 minutes, between 5 minutes and 45 minutes, between 5 minutes and 30 minutes, between 5 minutes and 15 minutes, between 15 minutes and 6 hours, between 15 minutes and 3 hours, between 15 minutes and 60 minutes, between 15 minutes and 45 minutes, between 15 minutes and 30 minutes, between 30 minutes and 6 hours, between 30 minutes and 3 hours, between 30 minutes and 60 minutes, between 30 minutes and 45 minutes, between 45 minutes and 6 hours, between 45 minutes and 3 hours, between 45 minutes and 60 minutes, between 60 minutes and 6 hours, or between 60 minutes and 3 hours.

In some cases, after a sample is contacted with a capture agent, the capture agent can be allowed to incubate on the sample at a given temperature. A capture agent can be incubated on the sample at about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 35° C., about 40° C., about 45° C., about 50° C., or about 55° C. In some cases, a capture agent can be incubated at a temperature of at least 4° C., at least 5° C., at least 6° C., at least 7° C., at least 8° C., at least 9° C., at least 10° C., at least 11° C., at least 12° C., at least 13° C., at least 14° C., at least 15° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 35° C., at least 40° C., at least 45° C., at least 50° C., or at least 55° C. In some cases, a capture agent can be incubated at a temperature of no more than 4° C., no more than 5° C., no more than 6° C., no more than 7° C., no more than 8° C., no more than 9° C., no more than 10° C., no more than 11° C., no more than 12° C., no more than 13° C., no more than 14° C., no more than 15° C., no more than 16° C., no more than 17° C., no more than 18° C., no more than 19° C., no more than 20° C., no more than 21° C., no more than 22° C., no more than 23° C., no more than 24° C., no more than 25° C., no more than 26° C., no more than 27° C., no more than 28° C., no more than 29° C., no more than 30° C., no more than 35° C., no more than 40° C., no more than 45° C., no more than 50° C., or no more than 55° C. In some cases, a capture agent can be incubated at at a temperature between 4° C. and 55° C., between 4° C. and 50° C., between 4° C. and 45° C., between 4° C. and 40° C., between 4° C. and 35° C., between 4° C. and 30° C., between 4° C. and 25° C., between 4° C. and 20° C., between 4° C. and 15° C., between 4° C. and 10° C., between 10° C. and 55° C., between 10° C. and 50° C., between 10° C. and 45° C., between 10° C. and 40° C., between 10° C. and 35° C., between 10° C. and 30° C., between 10° C. and 25° C., between 10° C. and 20° C., between 10° C. and 15° C., between 15° C. and 55° C., between 15° C. and 50° C., between 15° C. and 45° C., between 15° C. and 40° C., between 15° C. and 35° C., between 15° C. and 30° C., between 15° C. and 25° C., between 15° C. and 20° C., between 20° C. and 55° C., between 20° C. and 50° C., between 20° C. and 45° C., between 20° C. and 40° C., between 20° C. and 35° C., between 20° C. and 30° C., between 20° C. and 25° C., between 25° C. and 55° C., between 25° C. and 50° C., between 25° C. and 45° C., between 25° C. and 40° C., between 25° C. and 35° C., between 25° C. and 30° C., between 30° C. and 55° C., between 30° C. and 50° C., between 30° C. and 45° C., between 30° C. and 40° C., between 30° C. and 35° C., between 35° C. and 55° C., between 35° C. and 50° C., between 35° C. and 45° C., between 35° C. and 40° C., between 40° C. and 55° C., between 40° C. and 50° C., between 40° C. and 45° C., between 45° C. and 55° C., between 45° C. and 50° C., or between 50° C. and 55° C.

In some cases, after a sample is contacted with a capture agent, excess capture agent can be washed away. In some cases, a wash step can be performed using a wash buffer. A wash buffer can be any buffer than can wash away excess capture agent without significantly impacting the sample, bound capture agent, or oligonucleotide bound to capture agent. In some cases, a wash buffer can comprise PBS, PBS-T, TBS, TBS-T water, saline, or Kreb's buffer.

In some cases, a wash buffer can comprise a blocking component. A blocking component found in a wash buffer can be a protein blocking component or a nucleic acid blocking component. For example, a wash buffer can comprise BSA (bovine serum albumin) as a protein blocking component. As another example, a wash buffer can comprise sheared salmon-DNA as a nucleic acid blocking component. In some cases, a wash buffer can comprise more than one blocking component. In some cases, a wash buffer can comprise 1, 2, 3, 4, 5, or more blocking components. Some wash buffers can comprise a combination of a protein blocking component and a nucleic acid blocking component. Blocking components can include any acceptable blocking component, including any blocking component or blocking agent described herein.

Excess capture agent can be washed away in one or a plurality of washes. In some cases, about 1, about 2, about 3, about 4, about 5, or about 6 washes can be performed. In some cases, at least 1, at least 2, at least 3, at least 4, at least 5, or at least 6 washes can be performed. In some cases, no more than 1, no more than 2, no more than 3, no more than 4, no more than 5, or no more than 6 washes can be performed. In some cases, between 1 and 6, between 1 and 5, between 1 and 4, between 1 and 3, between 1 and 2, between 2 and 6, between 2 and 5, between 2 and 4, between 2 and 3, between 3 and 6, between 3 and 5, between 3 and 4, between 4 and 6, between 4 and 5, or between 5 and 6 washes can be performed.

Each wash can last about 10 seconds, about 15 seconds, about 30 seconds, about 1 minute, about 2 minutes, about 3 minutes, about 4 minutes, about 5 minutes, about 10 minutes, or about 15 minutes. Each wash can last at least 10 seconds, at least 15 seconds, at least 30 seconds, at least 1 minute, at least 2 minutes, at least 3 minutes, at least 4 minutes, at least 5 minutes, at least 10 minutes, or at least 15 minutes. In some cases, a wash can last for less than 10 seconds. Each wash can last up to 10 seconds, up to 15 seconds, up to 30 seconds, up to 1 minute, up to 2 minutes, up to 3 minutes, up to 4 minutes, up to 5 minutes, up to 10 minutes, or up to 15 minutes. In some cases, a wash can last for more than 15 minutes. Each wash can be between 10 seconds and 15 minutes, between 10 seconds and 10 minutes, between 10 seconds and 5 minutes, between 10 seconds and 1 minute, between 10 seconds and 30 seconds, between 30 seconds and 15 minutes, between 30 seconds and 10 minutes, between 30 seconds and 5 minutes, between 30 seconds and 1 minute, between 1 minute and 15 minutes, between 1 minute and 10 minutes, between 1 minute and 5 minutes, between 5 minutes and 15 minutes, between 5 minutes and 10 minutes, or between 10 minutes and 15 minutes.

Washes can be at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 35° C., about 40° C., about 45° C., about 50° C., or about 55° C. In some cases, washes can be at a temperature of at least 4° C., at least 5° C., at least 6° C., at least 7° C., at least 8° C., at least 9° C., at least 10° C., at least 11° C., at least 12° C., at least 13° C., at least 14° C., at least 15° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 35° C., at least 40° C., at least 45° C., at least 50° C., or at least 55° C. In some cases, washes can be at a temperature of no more than 4° C., no more than 5° C., no more than 6° C., no more than 7° C., no more than 8° C., no more than 9° C., no more than 10° C., no more than 11° C., no more than 12° C., no more than 13° C., no more than 14° C., no more than 15° C., no more than 16° C., no more than 17° C., no more than 18° C., no more than 19° C., no more than 20° C., no more than 21° C., no more than 22° C., no more than 23° C., no more than 24° C., no more than 25° C., no more than 26° C., no more than 27° C., no more than 28° C., no more than 29° C., no more than 30° C., no more than 35° C., no more than 40° C., no more than 45° C., no more than 50° C., or no more than 55° C. In some cases, washes can be at a temperature between 4° C. and 55° C., between 4° C. and 50° C., between 4° C. and 45° C., between 4° C. and 40° C., between 4° C. and 35° C., between 4° C. and 30° C., between 4° C. and 25° C., between 4° C. and 20° C., between 4° C. and 15° C., between 4° C. and 10° C., between 10° C. and 55° C., between 10° C. and 50° C., between 10° C. and 45° C., between 10° C. and 40° C., between 10° C. and 35° C., between 10° C. and 30° C., between 10° C. and 25° C., between 10° C. and 20° C., between 10° C. and 15° C., between 15° C. and 55° C., between 15° C. and 50° C., between 15° C. and 45° C., between 15° C. and 40° C., between 15° C. and 35° C., between 15° C. and 30° C., between 15° C. and 25° C., between 15° C. and 20° C., between 20° C. and 55° C., between 20° C. and 50° C., between 20° C. and 45° C., between 20° C. and 40° C., between 20° C. and 35° C., between 20° C. and 30° C., between 20° C. and 25° C., between 25° C. and 55° C., between 25° C. and 50° C., between 25° C. and 45° C., between 25° C. and 40° C., between 25° C. and 35° C., between 25° C. and 30° C., between 30° C. and 55° C., between 30° C. and 50° C., between 30° C. and 45° C., between 30° C. and 40° C., between 30° C. and 35° C., between 35° C. and 55° C., between 35° C. and 50° C., between 35° C. and 45° C., between 35° C. and 40° C., between 40° C. and 55° C., between 40° C. and 50° C., between 40° C. and 45° C., between 45° C. and 55° C., between 45° C. and 50° C., or between 50° C. and 55° C.

In some cases, upon contacting the biological sample, the antibody or antibody fragment can be bound to the element of the biological sample. The antibody or antibody fragment can bind reversibly or irreversibly with the element of the biological sample. Examples of how the antibody or antibody fragment can bind to the element of the biological sample can include ionic bonds or non-ionic bonds.

The antibody or antibody fragment can comprise an IgG, an IgM, a polyclonal antiboy, a monoclonal antibody, a scFv, a nanobody, a Fab, or a diabody. In some cases, the antibody or antibody fragment can be of mouse, rat, rabbit, human, camelid, or goat origin. In some cases, the antibody or antibody fragment can be raised against a human, mouse, rat, cow, pig, sheep, monkey, rabbit, fruit fly, frog, nematode or woodchuck antigen. In some cases, the antibody or antibody fragment can be raised against an animal, plant, bacteria, fungus, or protist antigen. In some cases, the antibody or antibody fragment can be raised against a virus, a viral vector, or a prion.

An antibody or antibody fragment can have sensitivity for an element of the sample. In some case, the element of the sample can comprise a protein, a DNA molecule, an RNA molecule, or a lipid. In some cases, sensitivity can refer to the fraction of elements correctly positively identified by the antibody or antibody fragment. The antibody or antibody fragment can have a sensitivity of at least 60%, at least 65%, at least 70%, at least 75%, at least 80%, at least 85%, at least 90%, at least 95%, or at least 99%.

An antibody or antibody fragment can have specificity for an element of the sample. In some case, the element of the sample can comprise a protein, a DNA molecule, an RNA molecule, or a lipid. In some cases, specificity can refer to the preference for an antibody or antibody fragment to bind to a given element over other element. The antibody or antibody fragment can have a specificity of at least 60%, at least 65%, at least 70%, at least 75%, at least 80%, at least 85%, at least 90%, at least 95%, or at least 99%.

An antibody or antibody fragment can have affinity for an element of the sample. In some cases, affinity can refer to how fast or how strong the antibody can bind to the element. Affinity can sometimes be described by the dissociation constant (Kd). The antibody or antibody fragment can have an affinity of no more than 10⁻⁴M, no more than 10⁻⁵M, no more than 10⁻⁶ M, no more than 10⁻⁷M, no more than 10⁻⁸ M, no more than 10⁻⁹ M, no more than 10⁻¹⁰ M, no more than 10⁻¹¹M, no more than 10⁻¹² M, no more than 10⁻¹³M, or no more than 10⁻¹⁴ M.

The antibody or antibody fragment can bind to at least 20%, 30%, 40%, 50%, 60%, 70%, 80%, or 90% of the element of the biological sample. In some cases, at least 50%, 60%, 70%, 80%, or 90% of antibody which is bound after contact with the biological sample can be bound to the element of the biological sample. In some cases, a non-specific bound antibody comprises less than 1%, less than 2%, less than 3%, less than 4%, less than 5%, less than 10%, less than 15%, less than 20%, less than 25%, less than 30%, less than 35%, or less than 40% of said antibody bound to the sample In some cases, the antibody not bound to the sample can be washed away after up to a 1, 2, 3, 4, 5, 10, 20, 30, 40, 50, or 60 minute incubation. In some cases, the antibody not bound to the sample can be washed away after over a 1, 2, 3, 4, 5, 6, 7, 8, 9, 10, 11, or 12 hour incubation. In some cases, the antibody not bound to the sample can be washed away after over a 0.5, 1, 2, 3, 4 or 5 day long incubation.

### Fixation and Crosslinking.

Capture agents can be fixed to the sample. In some cases, only capture agents which are bound to a biological feature of interest can be fixed to a sample. In some cases, all capture agents which are bound to a biological feature of interest can be fixed to a sample. Fixation of capture agents to a sample can be performed in some cases after excess (e.g., unbound) capture agent is washed away.

In some embodiments, the capture agents may be cross-linked or fixed to the sample, thereby preventing the capture agent from disassociating during subsequent steps. In some cases, cross-linking can prevent a capture agent from dissociating during an RCA reaction or during inactivation or removal of one or more labels. Thus, fixation or cross-linking of the capture agent to the sample can allow for an RCA reaction to be performed on the sample (rather than in solution), and can allow for multiplexing of the assay by permitting multiple iterations of reading to be performed, as the labels can be removed or inactivated without disturbing the capture agents. This crosslinking step may be done using any amine-to-amine crosslinker (e.g. formaldehyde, disuccinimiyllutarate or another reagent of similar action) although a variety of other chemistries can be used to cross-link the capture agent to the sample if desired.

### Oligonucleotides

An oligonucleotide can be a molecule which can be a chain of nucleotides. Methods, kits, and compositions herein can comprise three oligonucleotides. In some cases, the first oligonucleotide can contact the second oligonucleotide, while the second oligonucleotide can contact a third oligonucleotide. Oligonucleotides described herein can comprise ribonucleic acids. Oligonucleotides described herein can comprise deoxyribonucleic acids. In some cases, oligonucleotides can be any sequence, including a user-specified sequence. A portion of a sequence such as a user specified sequence can be designed such that it can be complimentary to another oligonucleotide. In some cases, three oligonucleotides can be user designed such that a portion of a first oligonucleotide can be complimentary to a first portion of a second oligonucleotide, and a second portion of the second oligonucleotide can be complimentary to a portion of a third oligonucleotide.

Sometimes, an oligonucleotide can comprise the nucleic acid bases G, A, T, C, U, or a combination thereof, or bases that are capable of base pairing reliably with a complementary nucleotide. 7-deaza-adenine, 7-deaza-guanine, adenine, guanine, cytosine, thymine, uracil, 2-deaza-2-thio-guanosine, 2-thio-7-deaza-guanosine, 2-thio-adenine, 2-thio-7-deaza-adenine, isoguanine, 7-deaza-guanine, 5,6-dihydrouridine, 5,6-dihydrothymine, xanthine, 7-deaza-xanthine, hypoxanthine, 7-deaza-xanthine, 2,6 diamino-7-deaza purine, 5-methyl-cytosine, 5-propynyl-uridine, 5-propynyl-cytidine, 2-thio-thymine or 2-thio-uridine are examples of such bases, although many others are known. An oligonucleotide may be an LNA, a PNA, a UNA, or an morpholino oligomer, for example. The oligonucleotides used herein may contain natural or non-natural nucleotides or linkages.

Herein, a capture agent such as an antibody or antibody fragment can be conjugated to a first oligonucleotide, such that at least a portion of the antibody or antibody fragment is in contact with an element of the biological sample. The first oligonucleotide can then contact a first binding region of a second oligonucleotide. A second binding region of the second oligonucleotide can then contact a third oligonucleotide, wherein the third oligonucleotide can comprise a detection component.

In some cases, an oligonucleotide can be conjugated or bound to a capture agent directly using any suitable chemical moiety on the capture agent. In some cases, an oligonucleotide can be linked to a capture agent enzymatically, e.g., by ligation. In some cases, an oligonucleotide can be linked indirectly to a capture agent, for example via a non-covalent interaction such as a biotin/streptavidin interaction or an equivalent thereof, via an aptamer or secondary antibody, or via a protein-protein interaction such as a leucine-zipper tag interaction or the like.

In some cases, an oligonucleotide can be bound to a capture agent using click chemistry, or a similar method. Click chemistry can refer to a class of biocompatible small molecule reactions that can allow the joining of molecules, such as an oligonucleotide and a capture agent. A click reaction can be a one pot reaction, and in some cases is not disturbed by water. A click reaction can generate minimal byproducts, non-harmful byproducts, or no byproducts. A click reaction can be driven by a large thermodynamic force. In some cases, a click reaction can be driven quickly and/or irreversibly to a high yield of a single reaction product (e.g., oligonucleotide conjugated to capture agent), and can have high reaction specificity. Click reactions can include but are not limited to [3+2] cycloadditions, thiol-ene reactions, Diels-Alder reactions, inverse electron demand Diels-Alder reactions, [4+1] cycloadditions, nucleophilic substitutions, carbonyl-chemistry-like formation of ureas, or addition reactions to carbon-carbon double bonds (e.g., dihydroxylation).

In some cases, a first oligonucleotide can comprise a plurality of ribonucleic acids (RNA). In some cases, the first oligonucleotide can comprise a plurality of deoxyribonucleic acids (DNA). In selected cases, the first oligonucleotide can comprise one or more synthetic nucleotides. Examples of synthetic nucleotides may include RNA analogues or DNA analogues. Some synthetic nucleotides can comprise artificial nucleic acids, which may comprise peptide nucleic acid, morpholino and locked nucleic acid, glycol nucleic acid, or threose nucleic acid.

The first oligonucleotide can have a given length appropriate for the application. In some cases, a longer oligonucleotide may be selected. In some cases, a shorter oligonucleotide may be selected. In some cases, pros and cons may be assessed when selecting oligonucleotide length, wherein the pros and cons may comprise melting temperature, secondary structure, a tertiary structure, affinity, specificity, selectivity, cost, or number of possible barcodes.

In some cases, the first oligonucleotide can be at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

In some cases, the first oligonucleotide can be between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

In some cases, the first oligonucleotide can be no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

In some cases, the first oligonucleotide can be wholly single stranded. In some cases, the first oligonucleotide can be partially double stranded. In some cases, the partially double stranded region can be at the 3′ end of the nucleotide, at the 5′ end of the nucleotide, or between the 5′ end and 3′ end of the nucleotide. In some cases, there may be more than one double stranded region. Some first oligonucleotides may have a secondary structure, a tertiary structure. Some first oligonucleotides may have a secondary structure such that the folding of a single strand and/or its complementarity to itself can produce one or more double stranded regions comprising a single strand.

A second oligonucleotide can contact the first oligonucleotide at a first binding region of the second oligonucleotide. This interaction can occur via base pairing.

The first binding region of the second oligonucleotide can be complimentary to at least a portion of said first oligonucleotide. In some cases, the first binding region can be complimentary to the 3′ end of the first oligonucleotide. In some cases, the first binding region can be complimentary to the 5′ end of the first oligonucleotide. In some cases, the first binding region can be complimentary to a region between the 3′ end and 5′ end of the first oligonucleotide. In some cases, the first binding region can be complimentary to the entire oligonucleotide. In some cases, the first binding region can be complimentary to less than 100% of the first oligonucleotide.

In some cases, such a first binding region can be capable of hybridizing with a first oligonucleotide. In some cases, such a first binding region can be complimentary to at least a portion of said first oligonucleotide. In some cases, such a first binding region can be at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some cases, such a first binding region can be between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some cases, such a first binding region can be no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some cases, such a first binding region can comprise one or more synthetic nucleotides.

In some cases, the second oligonucleotide can be at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

In some cases, the second oligonucleotide can be between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

In some cases, the second oligonucleotide can be no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

In some cases, the second oligonucleotide can be wholly single stranded. In some cases, the first oligonucleotide can be partially double stranded. In some cases, the partially double stranded region can be at the 3′ end of the nucleotide, at the 5′ end of the nucleotide, or between the 5′ end and 3′ end of the nucleotide. In some cases, there may be more than one double stranded region. Some second oligonucleotides may have a secondary structure or a tertiary structure. Some second oligonucleotides may have a secondary structure such that the folding of a single strand and/or its complementarity to itself can produce one or more double stranded regions comprising a single strand. In some cases, a second oligonucleotide can comprise more than one oligonucleotide. In some cases, a chain of oligonucleotides may be formed, connecting the first oligonucleotide with a third oligonucleotide.

A third oligonucleotide can contact the second oligonucleotide at a second binding region of the second oligonucleotide. This interaction can occur via base pairing.

The second binding region of the second oligonucleotide can be complimentary to at least a portion of said third oligonucleotide. In some cases, the second binding region can be complimentary to the 3′ end of the third oligonucleotide. In some cases, the second binding region can be complimentary to the 5′ end of the third oligonucleotide. In some cases, the second binding region can be complimentary to a region between the 3′ end and 5′ end of the third oligonucleotide. In some cases, the second binding region can be complimentary to the entire third oligonucleotide. In some cases, the second binding region can be complimentary to less than 100% of the third oligonucleotide.

In some cases, such a second binding region can be capable of hybridizing with a first oligonucleotide. In some cases, such a second binding region can be complimentary to at least a portion of said first oligonucleotide. In some cases, such a second binding region can be at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some cases, such a second binding region can be between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some cases, such a second binding region can be no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long. In some cases, such a second binding region can comprise one or more synthetic nucleotides.

In some cases, the third oligonucleotide can be at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

In some cases, the third oligonucleotide can be between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

In some cases, the third oligonucleotide can be no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

In some cases, the third oligonucleotide can be wholly single stranded. In some cases, the third oligonucleotide can be partially double stranded. In some cases, the partially double stranded region can be at the 3′ end of the nucleotide, at the 5′ end of the nucleotide, or between the 5′ end and 3′ end of the nucleotide. In some cases, there may be more than one double stranded region. Some third oligonucleotides may have a secondary structure. Some third oligonucleotides may have a secondary structure such that the folding of a single strand and/or its complementarity to itself can produce one or more double stranded regions comprising a single strand. In some cases, a second oligonucleotide can comprise more than one oligonucleotide. In some cases, a chain of oligonucleotides may be formed, connecting the first oligonucleotide with a third oligonucleotide.

### Detection Components

As described herein, a detection component can be affixed to the last oligonucleotide. Examples of detection components can include for example labels. A detection component can be a fluorophore, a radioisotope, a molecule capable of a colorimetric reaction, or a magnetic particle.

In some embodiments, a signal detected may be generated by fluorescence resonance energy transfer (FRET) and in other embodiments the detection may be done by raman spectroscopy, infrared detection, or magnetic/electrical detection. In some embodiments, the detecting step may involve a secondary nucleic acid amplification step, including, but not limited, to hybridization chain reaction, branched DNA (bDNA) amplification, etc.

In some cases, a detection component can be removed. Removal can be accomplished by washing, by cleaving, by an enzymatic reaction, by degrading, by chemically altering, or by other means.

Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), and POPR03 and TOPR03 (Molecular Probes, Eugene, Oreg.). Further suitable distinguishable detectable labels may be found in Kricka et al. (Ann Clin Biochem. 39:114-29, 2002), Ried et al. (Proc. Natl. Acad. Sci. 1992: 89: 1388-1392) and Tanke et al. (Eur. J. Hum. Genet. 1999 7:2-11) and others. In some embodiments three or four distinguishable dyes may be used. Specific fluorescent dyes of interest include: xanthene dyes, e.g., fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′, 5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g., Cy3, Cy5 and Cy7 dyes; coumarins, e.g., umbelliferone; benzimide dyes, e.g. Hoechst 33258; phenanthridine dyes, e.g., Texas Red; ethidium dyes; acridine dyes; carbazole dyes; phenoxazine dyes; porphyrin dyes; polymethine dyes, e.g., BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, RI 10, Eosin, JOE, R6G, Tetramethylrhodamine, TAMRA, Lissamine, Napthofluorescein, Texas Red, Cy3, and Cy5, etc. In some embodiments, within each sub-set of probes, the fluorophores may be chosen so that they are distinguishable, i.e., independently detectable, from one another, meaning that the labels can be independently detected and measured, even when the labels are mixed. In other words, the amounts of label present (e.g., the amount of fluorescence) for each of the labels are separately determinable, even when the labels are co-located (e.g., in the same tube or in the same area of the section).

The label may be a pro-fluorophore, a secondary activatable fluorophore, a fluorescent protein, a visible stain, a polychromatic barcode, a mass tag (e.g., an isotope or a polymer of a defined size), a structural tags for label-free detection, a radio sensitive tag (activated by THz camera) a radioactive tag or an absorbance tag that only absorbs light at a specific frequency for example. In some embodiments, an oligonucleotide may deliver an enzyme that delivers a fluorophore or there may be an enzymatic amplification of signal. In some cases, detectable signal of a label can be generated in some cases by fluorescence resonance energy transfer (FRET), Raman spectroscopy, infrared detection, or magnetic/electrical detection.

In some cases, a detection component can be the enzyme horseradish peroxidase (HRP). HRP can be an enzyme which can be found in the roots of horseradish. HRP can be a metalloenzyme. In some cases, HRP can present in one of a number of isoforms. Sometimes, the isoform can be type C.

If the detection component is HRP, detection can be accomplished by detecting the enzyme activity of the HRP. In some cases, this can be accomplished by exposing the HRP to a substrate, which may be an organic substrate, which may be able to be oxidized. In some cases, the substrate can be oxidized by the HRP, and sometimes the oxidized substrate can be detected. In some embodiments, the oxidation of a substrate which can be an organic substrate can be by hydrogen peroxide, wherein the HRP can catalyze the oxidation of the substrate by hydrogen peroxide. In some instances, the detection can be visual, or spectrophotometric, or performed using a camera, or by other detection means.

In some cases, a material mimicking natural HRP can be the detection component. For example, an HRP-like artificial enzyme can be used. For example, a material mimicking HRP can be an iron oxide nanoparticle or a hemin-containing complex.

Detection components can be located at any location on the last oligonucleotide. In some cases, a detection component can be at the 3′ end of the last oligonucleotide. In some cases, a detection component can be at the 4′ end of the last oligonucleotide. In some cases, a detection component can be between the 3′ and 4′ ends of the last oligonucleotide.

In some cases, wherein the last oligonucleotide comprises a detection component which is a fluorophore, the last oligonucleotide can additionally comprise a quencher. In some cases, the last oligonucleotide can have a secondary structure when unbound, wherein the secondary structure may bring the quencher near the fluorophore. The proximity of a quencher to a fluorophore can prevent detection of a signal from the fluorophore. Upon base pairing with a second oligonucleotide, the last oligonucleotide can experience a conformational change, which may spatially separate the quencher from the fluorophore, which may allow for detection of a fluorescent signal originating from the fluorophore.

Linkers

In compositions herein, different molecules can be connected via one or more linkers. For example, a capture agent can be attached to an oligonucleotide via a linker.

Linkers can comprise a direct bond or an atom such as oxygen or sulfur, a unit such as NR1, C(O), C(O)NH, SO, SO2, SO2NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO2, N(R1)2, C(O), cleavable linking group, substituted or unsubstituted aryl, substituted or unsubstituted heteroaryl, substituted or unsubstituted heterocyclic; where R1 is hydrogen, acyl, aliphatic or substituted aliphatic.

In some cases, the linker can be a nucleic acid linker. A “nucleic acid linker” can be a nucleic acid that connects two parts of a compound, e.g., an affinity molecule to a label moiety. A nucleic acid linker can be single-stranded, fully double-stranded, or partially double-stranded. A nucleic acid linker can be any length. For example, a nucleic acid linker can be from 1 nucleotide to about 100 nucleotides in length. When the nucleic acid linker is double-stranded, the linker can comprise a double stranded region of about 6 to about 100 consecutive base pairs. However, the duplex region can be interrupted by one or more single-stranded regions in one or both of the strands of the duplex. Further, a double-stranded nucleic acid linker can comprise a single-stranded overhang on one or both ends of the double-stranded region. Moreover, a nucleic acid linker can comprise one or more nucleic acid modifications described herein. A nucleic acid linker can be attached to a compound by a non-nucleic acid linker.

In some cases, a linker can be a “non-nucleic acid linker” which can be any linker that is not a nucleic acid linker.

A linker can link molecules covalently or non-covalently. Accordingly, in some embodiments, the capture agent and the oligonucleotide can be covalently linked together using a non-nucleic acid linker. For example, the capture agent and the oligonucleotide can be covalently linked together via a linker selected from the group consisting of a bond, succinimidyl-4-(N-maleimidomethyl)cyclohexane-1-carboxylate (SMCC) linker, sulfo-SMCC linker, succinimidyl-6-hydrazino-nicotinamide (S-HyNic) linker, N-succinimidyl-4-formylbenzamide (S-4FB) linker, bis-aryl hydrazone bond (from S-HyNic/S-4FB reaction), zero-length peptide bond (between —COOH and —NH₂ directly on affinity molecule and nucleic acid), two peptide bonds on a spacer (from cross-linking of two —NH₂ groups), triazole bond (from “click” reaction), a phosphodiester linkage, a phsophothioate linkage, and any combination thereof. In another example, the probe and the label can be covalently linked together via a linker selected from the group consisting of a bond, succinimidyl-4-(N-maleimidomethyl)cyclohexane-1-carboxylate (SMCC) linker, sulfo-SMCC linker, succinimidyl-6-hydrazino-nicotinamide (S-HyNic) linker, N-succinimidyl-4-formylbenzamide (S-4FB) linker, bis-aryl hydrazone bond (from S-HyNic/S-4FB reaction), zero-length peptide bond (between —COOH and —NH₂ directly on affinity molecule and nucleic acid), two peptide bonds on a spacer (from cross-linking of two —NH₂ groups), triazole bond (from “click” reaction), a phosphodiester linkage, a phsophothioate linkage, and any combination thereof.

### Experimental Conditions and Methods

A sample outline of an experimental workflow can comprise contacting a biological sample with an antibody or antibody fragment that is conjugated to a first oligonucleotide, contacting the first oligonucleotide with a first binding region of a second oligonucleotide, contacting a second binding region of said second oligonucleotide with a third oligonucleotide, wherein said third oligonucleotide comprises a detection component, thereby connectively coupling said biological sample to said detection component. The experimental workflow can be sequential, or in some cases steps may be combined.

A biological sample can be procured or prepared prior to or as part of methods described herein. Non-limiting examples of biological samples can include tissue, cells, or organs

In some cases, a protein blocking agent can be applied to the sample prior to the application of the capture agent.

A capture agent (or a plurality of capture agents) can be incubated on the sample. The capture agents can be linked to oligonucleotides, such that each capture agent is linked to a different oligonucleotide, as described herein. In some cases, one capture agent at a time can be incubated with the sample at the same time. In some cases, 2, 3, 4, 5, 6, 7, 8, or more capture agents can be incubated with the sample at the same time. In some cases, all capture agents can be incubated with the sample at the same time.

A capture agent can be incubated for about 1 minute, about 2 minutes, about 5 minutes, about 10 minutes, about 20 minutes, about 30 minutes, about 1 hour, about 2 hours, about 3 hours, about 4 hours, about 5 hours, or about 6 hours. In some cases, a capture agent can be incubated for at least 1 minute, at least 2 minutes, at least 5 minutes, at least 10 minutes, at least 20 minutes, at least 30 minutes, at least 1 hour, at least 2 hours, at least 3 hours, at least 4 hours, at least 5 hours, or at least 6 hours. In some cases, a capture agent can be incubated for not more than 1 minute, not more than 2 minutes, not more than 5 minutes, not more than 10 minutes, not more than 20 minutes, not more than 30 minutes, not more than 1 hour, not more than 2 hours, not more than 3 hours, not more than 4 hours, not more than 5 hours, or not more than 6 hours. In some cases, a capture agent can be incubated for between 1 minute and 6 hours, between 1 minute and 5 hours, between 1 minute and 4 hours, between 1 minute and 3 hours, between 1 minute and 2 hours, between 1 minute and 1 hour, between 1 minute and 30 minutes, between 1 minute and 20 minutes, between 1 minute and 10 minutes, between 1 minute and 5 minutes, between 1 minute and 2 minutes, between 2 minutes and 6 hours, between 2 minutes and 5 hours, between 2 minutes and 4 hours, between 2 minutes and 3 hours, between 2 minutes and 2 hours, between 2 minutes and 1 hour, between 2 minutes and 30 minutes, between 2 minutes and 20 minutes, between 2 minutes and 10 minutes, between 2 minutes and 5 minutes, between 5 minutes and 6 hours, between 5 minutes and 5 hours, between 5 minutes and 4 hours, between 5 minutes and 3 hours, between 5 minutes and 2 hours, between 5 minutes and 1 hour, between 5 minutes and 30 minutes, between 5 minutes and 20 minutes, between 5 minutes and 10 minutes, between 10 minutes and 6 hours, between 10 minutes and 5 hours, between 10 minutes and 4 hours, between 10 minutes and 3 hours, between 10 minutes and 2 hours, between 10 minutes and 1 hour, between 10 minutes and 30 minutes, between 10 minutes and 20 minutes, between 20 minutes and 6 hours, between 20 minutes and 5 hours, between 20 minutes and 4 hours, between 20 minutes and 3 hours, between 20 minutes and 2 hours, between 20 minutes and 1 hour, between 20 minutes and 30 minutes, between 30 minutes and 6 hours, between 30 minutes and 5 hours, between 30 minutes and 4 hours, between 30 minutes and 3 hours, between 30 minutes and 2 hours, between 30 minutes and 1 hour, between 1 hour and 6 hours, between 1 hour and 5 hours, between 1 hour and 4 hours, between 1 hour and 3 hours, between 1 hour and 2 hours, between 2 hours and 6 hours, between 2 hours and 5 hours, between 2 hours and 4 hours, between 2 hours and 3 hours, between 3 hours and 6 hours, between 3 hours and 5 hours, between 3 hours and 4 hours, between 4 hours and 6 hours, between 4 hours and 5 hours, or between 5 hours and 6 hours.

Following incubation with the capture agent, the sample can be washed to remove excess capture agent. Washing can comprise applying a buffer to the sample for an amount of time followed by removal of the buffer. In some cases, washing can comprise gentle agitation, such as by swirling, shaking, swinging, or rocking the sample. Washing can comprise applying at least 50 μL, at least 100 μL, at least 500 μL, at least 1 mL, at least 5 mL, at least 10 mL, at least 20 mL, at least 30 mL, at least 40 mL, or at least 50 mL buffer to the sample. Washing can comprise applying no more than 50 μL, no more than 100 μL, no more than 500 μL, no more than 1 mL, no more than 5 mL, no more than 10 mL, no more than 20 mL, no more than 30 mL, no more than 40 mL, or no more than 50 mL buffer to the sample. In some cases, washing can comprise applying between 50 μL, and 50 mL, between 50 μL, and 40 mL, between 50 μL, and 30 mL, between 50 μL, and 20 mL, between 50 μL, and 10 mL, between 50 μL, and 5 mL, between 50 μL, and 1 mL, between 50 μL, and 500 μL, between 50 μL, and 100 μL, between 100 μL, and 50 mL, between 100 μL, and 40 mL, between 100 μL, and 30 mL, between 100 μL and 20 mL, between 100 μL and 10 mL, between 100 μL and 5 mL, between 100 μL and 1 mL, between 100 μL and 500 μL, between 500 μL and 50 mL, between 500 μL and 40 mL, between 500 μL and 30 mL, between 500 μL and 20 mL, between 500 μL and 10 mL, between 500 μL and 5 mL, between 500 μL and 1 mL, between 1 mL and 50 mL, between 1 mL and 40 mL, between 1 mL and 30 mL, between 1 mL and 20 mL, between 1 mL and 10 mL, between 1 mL and 5 mL, between 5 mL and 50 mL, between 5 mL and 40 mL, between 5 mL and 30 mL, between 5 mL and 20 mL, between 5 mL and 10 mL, between 10 mL and 50 mL, between 10 mL and 40 mL, between 10 mL and 30 mL, between 10 mL and 20 mL, between 20 mL and 50 mL, between 20 mL and 40 mL, between 20 mL and 30 mL, between 30 mL and 50 mL, between 30 mL and 40 mL, or between 40 mL and 50 mL buffer to the sample. Wash buffer can be any acceptable buffer. In some cases, wash buffer can be for example a same buffer that the capture agent is in, or another buffer, such as PBS, PBS-T, TBS, or TBS-T. The washing step can last for at least 10 seconds, at least 30 seconds, at least 1 minute, at least 2 minutes, at least 3 minutes, at least 4 minutes, at least 5 minutes, at least 10 minutes, or at least 15 minutes. The washing step can last for up to 10 seconds, up to 30 seconds, up to 1 minute, up to 2 minutes, up to 3 minutes, up to 4 minutes, up to 5 minutes, up to 10 minutes, or up to 15 minutes. The washing step can last between 10 seconds and 15 minutes, between 10 seconds and 10 minutes, between 10 seconds and 5 minutes, between 10 seconds and 30 seconds, between 30 seconds and 15 minutes, between 30 seconds and 10 minutes, between 30 seconds and 5 minutes, between 30 seconds and 1 minute, between 1 minute and 15 minutes, between 1 minute and 10 minutes, between 1 minute and 5 minutes, between 5 minutes and 15 minutes, between 5 minutes and 10 minutes, or between 1 minutes and 15 minutes. A washing step can be performed 1, 2, 3, 4, 5, or more times.

A capture agent can be cross-linked to the sample. Such cross-linking can prevent the capture agent from disassociating during subsequent steps. Such a cross-linking step may be done using any amine-to-amine crosslinker (e.g. formaldehyde, paraformaldehyde, disuccinimiyllutarate, N-hydroxysuccinimide (NHS), or another reagent of similar action) although a variety of other chemistries can be used to cross-link the capture agent to the sample if desired.

In some cases, a nucleic acid blocking agent can be applied to the sample. Any acceptable nucleic acid blocking agent can be used in this step, such as salmon sperm DNA or another commercially available product.

In some cases, a nucleic acid blocking agent can be incubated at about 4° C., about 10° C., about 15° C., about 20° C., about 25° C., about 30° C., about 35° C., about 40° C., or about 45° C. In some cases, a nucleic acid blocking agent can be incubated at at least 4° C., at least 10° C., at least 15° C., at least 20° C., at least 25° C., at least 30° C., at least 35° C., at least 40° C., or at least 45° C. In some cases, a nucleic acid blocking agent can be incubated at not more than 4° C., not more than 10° C., not more than 15° C., not more than 20° C., not more than 25° C., not more than 30° C., not more than 35° C., not more than 40° C., or not more than 45° C. In some cases, a nucleic acid blocking agent can be incubated between 4° C. and 45° C., between 4° C. and 40° C., between 4° C. and 35° C., between 4° C. and 30° C., between 4° C. and 25° C., between 4° C. and 20° C., between 4° C. and 15° C., between 4° C. and 10° C., between 10° C. and 45° C., between 10° C. and 40° C., between 10° C. and 35° C., between 10° C. and 30° C., between 10° C. and 25° C., between 10° C. and 20° C., between 10° C. and 15° C., between 15° C. and 45° C., between 15° C. and 40° C., between 15° C. and 35° C., between 15° C. and 30° C., between 15° C. and 25° C., between 15° C. and 20° C., between 20° C. and 45° C., between 20° C. and 40° C., between 20° C. and 35° C., between 20° C. and 30° C., between 20° C. and 25° C., between 25° C. and 45° C., between 25° C. and 40° C., between 25° C. and 35° C., between 25° C. and 30° C., between 30° C. and 45° C., between 30° C. and 40° C., between 30° C. and 35° C., between 35° C. and 45° C., between 35° C. and 40° C., or between 40° C. and 45° C.

In some cases, the blocking step can last for about 10 minutes, about 20 minutes, about 30 minutes, about 40 minutes, about 50 minutes, or about 60 minutes. In some cases, the blocking step can last for at least 10 minutes, at least 20 minutes, at least 30 minutes, at least 40 minutes, at least 50 minutes, or at least 60 minutes. In some cases, the blocking step can last for not more than 10 minutes, not more than 20 minutes, not more than 30 minutes, not more than 40 minutes, not more than 50 minutes, or not more than 60 minutes. In some cases, the blocking step can last for between 10 minutes and 60 minutes, between 10 minutes and 50 minutes, between 10 minutes and 40 minutes, between 10 minutes and 30 minutes, between 10 minutes and 20 minutes, between 20 minutes and 60 minutes, between 20 minutes and 50 minutes, between 20 minutes and 40 minutes, between 20 minutes and 30 minutes, between 30 minutes and 60 minutes, between 30 minutes and 50 minutes, between 30 minutes and 40 minutes, between 40 minutes and 60 minutes, between 40 minutes and 50 minutes, or between 50 minutes and 60 minutes.

The antibody or antibody fragment conjugated to the first oligonucleotide can be in a first buffer.

A second oligonucleotide can be incubated on the sample, such that the second oligonucleotide has an opportunity to hybridize with the first oligonucleotide.

In some cases, a second oligonucleotide can be incubated at about 4° C., about 10° C., about 15° C., about 20° C., about 25° C., about 30° C., about 35° C., about 40° C., or about 45° C. In some cases, a second oligonucleotide can be incubated at at least 4° C., at least 10° C., at least 15° C., at least 20° C., at least 25° C., at least 30° C., at least 35° C., at least 40° C., or at least 45° C. In some cases, a second oligonucleotide can be incubated at not more than 4° C., not more than 10° C., not more than 15° C., not more than 20° C., not more than 25° C., not more than 30° C., not more than 35° C., not more than 40° C., or not more than 45° C. In some cases, a second oligonucleotide can be incubated between 4° C. and 45° C., between 4° C. and 40° C., between 4° C. and 35° C., between 4° C. and 30° C., between 4° C. and 25° C., between 4° C. and 20° C., between 4° C. and 15° C., between 4° C. and 10° C., between 10° C. and 45° C., between 10° C. and 40° C., between 10° C. and 35° C., between 10° C. and 30° C., between 10° C. and 25° C., between 10° C. and 20° C., between 10° C. and 15° C., between 15° C. and 45° C., between 15° C. and 40° C., between 15° C. and 35° C., between 15° C. and 30° C., between 15° C. and 25° C., between 15° C. and 20° C., between 20° C. and 45° C., between 20° C. and 40° C., between 20° C. and 35° C., between 20° C. and 30° C., between 20° C. and 25° C., between 25° C. and 45° C., between 25° C. and 40° C., between 25° C. and 35° C., between 25° C. and 30° C., between 30° C. and 45° C., between 30° C. and 40° C., between 30° C. and 35° C., between 35 t and 45° C., between 35 t and 40° C., or between 40° C. and 45° C.

In some cases, a second oligonucleotide can be incubated on the sample for about 10 minutes, about 20 minutes, about 30 minutes, about 40 minutes, about 50 minutes, or about 60 minutes. In some cases, a second oligonucleotide can be incubated on the sample for at least 10 minutes, at least 20 minutes, at least 30 minutes, at least 40 minutes, at least 50 minutes, or at least 60 minutes. In some cases, a second oligonucleotide can be incubated on the sample for not more than 10 minutes, not more than 20 minutes, not more than 30 minutes, not more than 40 minutes, not more than 50 minutes, or not more than 60 minutes. In some cases, a second oligonucleotide can be incubated on the sample for between 10 minutes and 60 minutes, between 10 minutes and 50 minutes, between 10 minutes and 40 minutes, between 10 minutes and 30 minutes, between 10 minutes and 20 minutes, between 20 minutes and 60 minutes, between 20 minutes and 50 minutes, between 20 minutes and 40 minutes, between 20 minutes and 30 minutes, between 30 minutes and 60 minutes, between 30 minutes and 50 minutes, between 30 minutes and 40 minutes, between 40 minutes and 60 minutes, between 40 minutes and 50 minutes, or between 50 minutes and 60 minutes.

The second oligonucleotide can be in a second buffer. In some cases, the second buffer can comprise PBS, PBS-T, TBS, TBS-T water, saline, or Kreb's buffer.

A third oligonucleotide can be incubated on the sample, such that the third oligonucleotide has an opportunity to hybridize with the second oligonucleotide.

In some cases, a third oligonucleotide can be incubated at about 4° C., about 10° C., about 15° C., about 20° C., about 25° C., about 30° C., about 35° C., about 40° C., or about 45° C. In some cases, a third oligonucleotide can be incubated at at least 4° C., at least 10° C., at least 15° C., at least 20° C., at least 25° C., at least 30° C., at least 35° C., at least 40° C., or at least 45° C. In some cases, a third oligonucleotide can be incubated at not more than 4° C., not more than 10° C., not more than 15° C., not more than 20° C., not more than 25° C., not more than 30° C., not more than 35° C., not more than 40° C., or not more than 45° C. In some cases, a third oligonucleotide can be incubated between 4 t and 45° C., between 4 t and 40° C., between 4 t and 35° C., between 4 t and 30° C., between 4 t and 25° C., between 4° C. and 20° C., between 4° C. and 15° C., between 4° C. and 10° C., between 10° C. and 45° C., between 10° C. and 40° C., between 10° C. and 35° C., between 10° C. and 30° C., between 10° C. and 25° C., between 10° C. and 20° C., between 10° C. and 15° C., between 15° C. and 45° C., between 15° C. and 40° C., between 15 t and 35° C., between 15 t and 30° C., between 15 t and 25° C., between 15 t and 20° C., between 20° C. and 45° C., between 20° C. and 40° C., between 20 t and 35° C., between 20° C. and 30° C., between 20° C. and 25° C., between 25 t and 45° C., between 25° C. and 40° C., between 25° C. and 35° C., between 25° C. and 30° C., between 30° C. and 45° C., between 30° C. and 40° C., between 30° C. and 35° C., between 35° C. and 45° C., between 35° C. and 40° C., or between 40° C. and 45° C.

In some cases, a third oligonucleotide can be incubated on the sample for about 10 minutes, about 20 minutes, about 30 minutes, about 40 minutes, about 50 minutes, or about 60 minutes. In some cases, a third oligonucleotide can be incubated can be incubated on the sample for at least 10 minutes, at least 20 minutes, at least 30 minutes, at least 40 minutes, at least 50 minutes, or at least 60 minutes. In some cases, a third oligonucleotide can be incubated on the sample for not more than 10 minutes, not more than 20 minutes, not more than 30 minutes, not more than 40 minutes, not more than 50 minutes, or not more than 60 minutes. In some cases, a third oligonucleotide can be incubated on the sample for between 10 minutes and 60 minutes, between 10 minutes and 50 minutes, between 10 minutes and 40 minutes, between 10 minutes and 30 minutes, between 10 minutes and 20 minutes, between 20 minutes and 60 minutes, between 20 minutes and 50 minutes, between 20 minutes and 40 minutes, between 20 minutes and 30 minutes, between 30 minutes and 60 minutes, between 30 minutes and 50 minutes, between 30 minutes and 40 minutes, between 40 minutes and 60 minutes, between 40 minutes and 50 minutes, or between 50 minutes and 60 minutes.

The third oligonucleotide can be in a third buffer. In some cases, the third buffer can comprise PBS, PBS-T, TBS, TBS-T water, saline, or Kreb's buffer.

In some cases, the first buffer can be the same, or substantially the same, as the second buffer. In some cases, the second buffer can be the same, or substantially the same, as the third buffer. In some cases, the first buffer can be the same, or substantially the same, as the third buffer.

In some cases, the antibody conjugated to the first oligonucleotide can be in the same buffer as the second oligonucleotide, which can be a first alternate buffer. In some cases, the first alternate buffer can comprise PBS, PBS-T, TBS, TBS-T water, saline, or Kreb's buffer.

In some cases, the second oligonucleotide can be in the same buffer as the third oligonucleotide, which can be a second alternate buffer. In some cases, the second alternate buffer can comprise PBS, PBS-T, TBS, TBS-T water, saline, or Kreb's buffer

In some cases, the antibody conjugated to the first oligonucleotide, the second oligonucleotide, and the third oligonucleotide can be in the same buffer, which can be a common buffer. In some cases, the common buffer can comprise PBS, PBS-T, TBS, TBS-T water, saline, or Kreb's buffer.

A nonlimiting example protocol can proceed as follows. A volume of an antibody or antibody fragment conjugated to the first oligonucleotide in a first buffer can be layered onto a previously prepared sample. After an incubation time has elapsed, in some cases the volume can be washed off. In some instances, the buffer used for washing can comprise PBS, PBS-T, TBS, TBS-T water, saline, or Kreb's buffer. In some cases, a buffer used for washing can be the same buffer as the first buffer, but may not include the antibody or antibody fragment conjugated to the first oligonucleotide.

The incubation time for any step in a protocol similar to the above one can be up to 10, 20, 30, 40, 50, or 60 minutes. The incubation time for any step in a protocol similar to the above one can be at least 1, 2, 3, 4, 5, 6, 7, 8, 9, 10, 11, 12, 13, 14, 15, 16, 17, 18, 19, 20, 21, 22, 23, or 24 hours. The incubation for any step in a protocol similar to the above one can last about a workday, about overnight, about a weekend, about a week, or about a month.

For some applications, more than one antibody or antibody fragment can be used. In such cases, different antibodies or antibody fragments can be used. In some cases, at least 1, 2, 3, 4, 5, 6, 7, 8, 9, 10, 15, 20, 25, 30, or more different antibodies or antibody fragments can be used. Antibodies or antibody fragments can differ in sequence by up to 5%, 10%, 20%, 30%, 40%, 50%, 60%, 70%, 80%, 90%, or 95%. In some cases, antibodies or antibody fragments can differ by between 1% and 10%, between 1% and 20%, between 1% and 30%, between 1% and 40%, between 5% and 10%, between 5% and 20%, between 5% and 30%, between 5% and 40%, between 10% and 20%, between 10% and 30%, or between 30% and 40%.

In some cases, each different antibody or antibody fragment can be conjugated to a unique first oligonucleotide. Each first oligonucleotide can be different from other first oligonucleotides by at least 1, 2, 3, 4, 5, 6, 7, 8, 9, 10, 15, 20, 25, 30, 35, 40, 45, 50, 55, 60, 65, 70, 75, 80, 85, 90, 95, 100, or more base pairs. In some cases, a first oligonucleotide can be different from other first oligonucleotides by at least 1%, 5%, 10%, 20%, 25%, 30%, 35%, 40%, 45%, 50%, 55%, 60%, 65%, 70%, 75%, 80%, 85%, 90%, 95%, 99%, or 100%. Each first oligonucleotide can have at least 1, 2, 3, 4, 5, 6, 7, 8, 9, 10, 15, 20, 25, 30, 35, 40, 45, 50, 55, 60, 65, 70, 75, 80, 85, 90, 95, 100, or more base pairs which can be the same as in other first oligonucleotides. First oligonucleotides having base pairs which are the same as in other first oligonucleotides can have base pairs which are the same as in 1, 2, 3, 4, 5, 6, 7, 8, 9, 10, 11, 12, 13, 14, 15, 16, 17, 18, 19, 20, 30, 40, 50, 60, 70, 80, 90, 100, or more other first oligonucleotides.

Reading

A sample can be read to determine the binding pattern for one or more of the capture agents. In some cases, a sample can be read to determine the binding pattern for each of the capture agents. Such a binding pattern can indicate spatial information of an oligonucleotide and conjugated capture agent, which can in turn indicate spatial information of a biological feature of interest.

A method to determine such a binding pattern can comprise reading the sample to obtain an image from which the binding pattern for each of the sub-set of capture agents hybridized in the prior step can be determined (i.e., the binding patterns of different capture agents bound to different biological features). This step may be done using any convenient reading method and, in some embodiments, e.g., hybridization of the different probes can be separately read using a fluorescence microscope equipped with an appropriate filter for each fluorophore, or by using dual or triple band-pass filter sets to observe multiple fluorophores (see, e.g., U.S. Pat. No. 5,776,688).

In some cases, each biological feature of interest associated with a label at the same time another biological feature of interest is associated with a label can be read during the same iteration. Labels read during the same iteration can be different. Two labels can be considered different if they are distinguishable from each other when detected using the reading medium. For example, two fluorescent molecules can be considered different if when imaged using a microscope, their signals are differentiable from each other, e.g. by excitation wavelength, emission wavelength, intensity, or some other property.

Each reading can produce an image of the sample showing the pattern of binding of a sub-set of capture agents. In some embodiments, the method may further comprise analyzing, comparing or overlaying, at least two of the images. In some embodiments, the method may further comprise overlaying all of the images to produce an image showing the pattern of binding of all of the capture agents to the sample. The image analysis module used may transform the signals from each fluorophore to produce a plurality of false color images. The image analysis module may overlay the plurality of false color images (e.g., superimpose the false colors at each pixel) to obtain a multiplexed false color image. Multiple images (e.g., unweighted or weighted) may be transformed into a single false color, e.g., to represent a biological feature of interest characterized by the binding of a specific capture agent. False colors may be assigned to specific capture agents or combinations of capture agents, based on manual input from the user. In certain aspects, the image may comprise false colors relating only to the intensities of labels associated with a feature of interest, such as in the nuclear compartment. The image analysis module may further be configured to adjust (e.g., normalize) the intensity and/or contrast of signal intensities or false colors, to perform a convolution operation (such as blurring or sharpening of the intensities or false colors), or perform any other suitable operations to enhance the image. The image analysis module may perform any of the above operations to align pixels obtained from successive images and/or to blur or smooth intensities or false colors across pixels obtained from successive images.

In some embodiments, images of the sample may be taken at different focal planes, in the z direction. These optical sections can be used to reconstruct a three-dimensional image of the sample. Optical sections may be taken using confocal microscopy, although other methods are known. The image analysis method may be implemented on a computer. In certain embodiments, a general-purpose computer can be configured to a functional arrangement for the methods and programs disclosed herein. The hardware architecture of such a computer is well known by a person skilled in the art, and can comprise hardware components including one or more processors (CPU), a random-access memory (RAM), a read-only memory (ROM), an internal or external data storage medium (e.g., hard disk drive). A computer system can also comprise one or more graphic boards for processing and outputting graphical information to display means. The above components can be suitably interconnected via a bus inside the computer. The computer can further comprise suitable interfaces for communicating with general-purpose external components such as a monitor, keyboard, mouse, network, etc. In some embodiments, the computer can be capable of parallel processing or can be part of a network configured for parallel or distributive computing to increase the processing power for the present methods and programs. In some embodiments, the program code read out from the storage medium can be written into a memory provided in an expanded board inserted in the computer, or an expanded unit connected to the computer, and a CPU or the like provided in the expanded board or expanded unit can actually perform a part or all of the operations according to the instructions of the program code, so as to accomplish the functions described below. In other embodiments, the method can be performed using a cloud computing system. In these embodiments, the data files and the programming can be exported to a cloud computer, which runs the program, and returns an output to the user.

Inactivation and Removal

Labels can be inactivated or removed. Inactivation or removal can allow for multiplexing of the method, such that a greater plurality of biological features of interest can be detected than without an inactivation or removal step.

After reading the sample, the method may comprise inactivating or removing the labels that are associated with (i.e., hybridized to) an oligonucleotide leaving the plurality of capture agents and their associated oligonucleotides still bound to the sample. The labels that are associated the sample may be removed or inactivated by a variety of methods including, but not limited to, denaturation (in which case the label and the oligonucleotide it is bound to in its entirety can be released and can be washed away), by cleaving a linkage in the probe (in which case the label and part of an oligonucleotide can be released and can be washed away), by cleaving both an oligonucleotide that the label is bound to (third oligonucleotide) and the oligonucleotide it is hybridized to (second oligonucleotide) to release a fragment that can be washed away, or by cleaving the linkage between the oligonucleotide and the label (in which case the label can be released and can be washed away), by cleaving an oligonucleotide such as by using a restriction enzyme (in which case the oligonucleotide and the label can be washed away), or by inactivating the label itself (e.g., by breaking a bond in the label, thereby preventing the label from producing a signal, or by introducing a quencher to the label to prevent detection of a signal). In acceptable removal methods such as the ones provided, the unhybridized oligonucleotides that are attached to the other antibodies (e.g., antibodies bound to biological features of interest not yet detected) can remain intact and free to hybridize to the set of labeled probes used in the next cycle. In some embodiments, fluorescence may be inactivated by light-based bleaching, peroxide-based bleaching, or cleavage of a fluorophore linked to a nucleotide through a cleavable linker (e.g., using TCEP as a cleaving reagent).

In some embodiments, the removing step is done by removing the hybridized probes from the sample by denaturation, leaving the other capture agents and their associated oligonucleotides still bound to the sample. In other embodiments, the removing step is not done by denaturation, leaving the other capture agents (i.e., the capture agents that are not hybridized to a probe) and their associated oligonucleotides still bound to the sample. In these embodiments, the labels may be removed by cleaving at least one bond in the capture agent—three oligonucleotide—label complex that is associated with the sample, or a linker that links an oligonucleotide from a label, thereby releasing the labels from the oligonucleotide. This cleavage can be performed enzymatically, chemically or via exposure to light. Alternatively, the labels can be inactivated by photobleaching or by chemically altering the label.

If a removal step is not performed by removing a hybridized nucleotide from the sample by denaturation, then a variety of chemical-based, enzyme-catalyzed or photo-induced cleavage methods may be used. For example, in some embodiments, the oligonucleotide may contain a chemically or photo-cleavable linkage so that they can be fragmented by exposure to a chemical or light. In some embodiments, the duplexes (because they are double stranded) may be cleaved by a restriction enzyme or a double-stranded DNA specific endonuclease (a fragmentase), for example. In some embodiments, the oligonucleotide may contain a uracil residue (which can be cleaved by USER), or may contain a hairpin that contains a mismatch, which can be cleaved using a mismatch-specific endonuclease. In some of these embodiments, after cleavage the Tm of the fragment of the oligonucleotide that contains the label may be insufficiently high to remain base paired with the oligonucleotide and, as such, the fragment can disassociate from the oligonucleotide. In some embodiments, the oligonucleotide and the label may be connected by a photo-cleavable or chemically-cleavable linker. Cleavage of this linker can release the label from the sample. In other embodiments, the oligonucleotide may be an RNA, and the oligonucleotide can be degraded using an RNAse. In some embodiments, an enzymatically cleavable linkage can be used. For example, esters can be cleaved by an esterase and a glycan can be cleaved by a glycase. Alternatively, the label itself may be inactivated by modifying the label. In one example, the dye may be photobleached, but other methods are known.

In some embodiments, after reading the sample, the method may comprise removing the hybridized third oligonucleotide from the sample by denaturation (i.e., by un-annealing the labeled probes from the oligonucleotides and washing them away), leaving the capture agents and their associated oligonucleotides still bound to the sample. This step may be done using any suitable chemical denaturant, e.g., formamide, DMSO, urea, or a chaotropic agent (e.g., guanidinium chloride or the like), using a toehold release strategy (see, e.g., Kennedy-Darling, Chembiochem. 2014 15: 2353-2356), or using heat, base, a topoisomerase or a single-strand binding agent (e.g., SSBP). This step can also be achieved through hybridization of an oligonucleotide with a greater affinity (e.g. PNA). In some cases, the probes may by removed by incubating the sample in 70% to 90% formamide (e.g., 75% to 85% formamide) for a period of at least 1 minute (e.g., 1 to 5 minutes), followed by a wash. This denaturation step may be repeated, if necessary, so that all of the hybridized probes have been removed. As would be apparent, this step is not implemented enzymatically, i.e., does not use a nuclease such as a DNAse or a restriction enzyme, and does not result in cleavage of any covalent bonds, e.g., in any of the probes or oligonucleotides or removal of any of the capture agents from the sample. In this step, the strands of the probe/oligonucleotide duplexes are separated from one another (i.e., denatured), and the separated probes, which are now free in solution, are washed away, leaving the capture agents and their associated oligonucleotides intact and in place.

If a cleavable linkage is used (e.g., in the oligonucleotide or to connect an oligonucleotide to a label, then the cleavable linker can be capable of being selectively cleaved using a stimulus (e.g., light or a change in its environment) without breakage of bonds in the oligonucleotides attached to the antibodies. In some embodiments, the cleavable linkage may be a disulfide bond, which can be readily broken using a reducing agent (e.g., β-mercaptoethanol or another suitable reducing agent). Suitable cleavable bonds that may be employed include, but are not limited to, the following: base-cleavable sites such as esters, particularly succinates (cleavable by, for example, ammonia or trimethylamine), quaternary ammonium salts (cleavable by, for example, diisopropylamine) and urethanes (cleavable by aqueous sodium hydroxide); acid-cleavable sites such as benzyl alcohol derivatives (cleavable using trifluoroacetic acid), teicoplanin aglycone (cleavable by trifluoroacetic acid followed by base), acetals and thioacetals (also cleavable by trifluoroacetic acid), thioethers (cleavable, for example, by HF or cresol) and sulfonyls (cleavable by trifluoromethane sulfonic acid, trifluoroacetic acid, thioanisole, or the like); nucleophile-cleavable sites such as phthalamide (cleavable by substituted hydrazines), esters (cleavable by, for example, aluminum trichloride); and Weinreb amide (cleavable by lithium aluminum hydride); and other types of chemically cleavable sites, including phosphorothioate (cleavable by silver or mercuric ions) and diisopropyldialkoxysilyl (cleavable by fluoride ions). Other cleavable bonds can be apparent to those skilled in the art or are described in the pertinent literature and texts (e.g., Brown (1997) Contemporary Organic Synthesis 4(3); 216-237). A cleavable bond may be cleaved by an enzyme in some embodiments.

In particular embodiments, a photocleavable (“PC”) linker (e.g., a uv-cleavable linker) may be employed. Suitable photocleavable linkers for use may include ortho-nitrobenzyl-based linkers, phenacyl linkers, alkoxybenzoin linkers, chromium arene complex linkers, NpSSMpact linkers and pivaloylglycol linkers, as described in Guillier et al (Chem Rev. 2000 June 14; 100(6):2091-158). Exemplary linking groups that may be employed in the subject methods may be described in Guillier et al, supra and Olejnik et al. (Methods in Enzymology 1998 291:135-154), and further described in U.S. Pat. No. 6,027,890; Olejnik et al. (Proc. Natl. Acad Sci, 92:7590-94); Ogata et al. (Anal. Chem. 2002 74:4702-4708); Bai et al. (Nucl. Acids Res. 2004 32:535-541); Zhao et al. (Anal. Chem. 2002 74:4259-4268); and Sanford et al. (Chem Mater. 1998 10:1510-20), and are purchasable from Ambergen (Boston, Mass.; NHS-PC-LC-Biotin), Link Technologies (Bellshill, Scotland), Fisher Scientific (Pittsburgh, Pa.) and Calbiochem-Novabiochem Corp. (La Jolla, Calif.).

### Iterative Methods

Methods herein can comprise steps that are repeated. In some cases, this can comprise repeating steps of the method. This can allow for a greater plurality of biological features of interest to be detected than can be accomplished without repeating the steps.

After removal or inactivation of a label, the sample may be hybridized with a different set of labeled oligonucleotides (i.e., third oligonucleotide(s) or set(s) of second oligonucleotide(s) and third oligonucleotide(s)) comprising an additional label(s) and the sample may be re-read to produce an image showing the binding pattern for the capture agents associated with each of the most recently hybridized oligonucleotide(s). In this manner, in different iterations of reading the sample, different biological features of interest can be detected. After the sample has been read, the label(s) may be removed from the sample, e.g., by denaturation, inactivation, or another method (as described above), and the hybridization and reading steps may be repeated with another different set of distinguishably labeled oligonculeotides which can hybridize to oligonucleotides associated with another different subset of capture agents. In other words, the method may comprise repeating the hybridization, label removal or inactivation and reading steps multiple times with a different sub-set of the labeled oligonucleotides, where the probes in each sub-set can be distinguishably labeled and each repeat can be followed by removal of the labels, e.g., by denaturation or another method (except for the final repeat) to produce a plurality of images of the sample, where each image corresponds to a sub-set of labeled oligonucleotides. The hybridization/reading/label removal or inactivation steps can be repeated until desired biological features of interest have been analyzed.

Nucleotide sequences used may be selected in order to minimize background staining, either from non-specific adsorption or through binding to endogenous genomic sequences (RNA or DNA). Likewise, the hybridization and washing buffers may be designed to minimize background staining either from non-specific adsorption or through binding to endogenous genomic sequences (RNA or DNA) or through binding to other reporter sequences.

In addition to the labeling methods described above, the sample may be stained using a cytological stain, either before or after performing the method described above. In these embodiments, the stain may be, for example, phalloidin, gadodiamide, acridine orange, bismarck brown, barmine, Coomassie blue, bresyl violet, brystal violet, DAPI, hematoxylin, eosin, ethidium bromide, acid fuchsine, haematoxylin, hoechst stains, iodine, malachite green, methyl green, methylene blue, neutral red, Nile blue, Nile red, osmium tetroxide (formal name: osmium tetraoxide), rhodamine, safranin, phosphotungstic acid, osmium tetroxide, ruthenium tetroxide, ammonium molybdate, cadmium iodide, carbohydrazide, ferric chloride, hexamine, indium trichloride, lanthanum nitrate, lead acetate, lead citrate, lead(II) nitrate, periodic acid, phosphomolybdic acid, potassium ferricyanide, potassium ferrocyanide, ruthenium red, silver nitrate, silver proteinate, sodium chloroaurate, thallium nitrate, thiosemicarbazide, uranyl acetate, uranyl nitrate, vanadyl sulfate, or any derivative thereof. The stain may be specific for any feature of interest, such as a protein or class of proteins, phospholipids, DNA (e.g., dsDNA, ssDNA), RNA, an organelle (e.g., cell membrane, mitochondria, endoplasmic recticulum, golgi body, nuclear envelope, or other organelle), or a compartment of the cell (e.g., cytosol, nuclear fraction, or other compartment). The stain may enhance contrast or imaging of intracellular or extracellular structures. In some embodiments, the sample may be stained with haematoxylin and eosin (H&E).

### Kits

Also provided herein are kits that can contain reagents for practicing the methods described above. In some embodiments, the kit may comprise an antibody or antibody fragment which is conjugated to a first oligonucleotide, a second oligonucleotide comprising a first binding region and a second binding region, wherein the first binding region of said second oligonucleotide can be complimentary to at least a portion of said first oligonucleotide; and a third oligonucleotide comprising a detection component, wherein said second binding region of said second oligonucleotide can be complimentary to at least a portion of said third oligonucleotide. In some embodiments, the detection component can be pre-attached to the third oligonucleotide, and in other embodiments, the detection component can be attached to the third oligonucleotide at a later time.

A kit can comprise instructions for imaging a sample with the labeled probes bound. In some cases, such a kit can comprise instructions and/or reagents which allow multiplexing of the imaging protocol. For example, a kit can comprise instructions and/or reagents for chemically removing, inactivating, quenching, cleaving, or dehybridizing a label.

In the above-mentioned components, the kits may further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

In addition to above-mentioned components, a kit can further include instructions for using the components of the kit to practice the subject methods, i.e., instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment can be a kit that includes a web address where the instructions can be viewed and/or from which the instructions can be downloaded. As with the instructions, this means for obtaining the instructions is recorded on a suitable substrate.

### EXAMPLES

### Example 1: Element Detection in Issue Fixed on a Slide

In some embodiments, a biological sample, for example a slice of tissue or a plurality of cultured cells, can be fixed on a slide. Once on the slide, the sample can be contacted with an antibody or antibody fragment which can be conjugated to a first oligonucleotide. The oligonucleotide can be in volume of a suitable buffer which can be capable of maintaining the integrity of the antibody or antibody fragment, oligonucleotide, biological sample, and any interactions between the antibody or antibody fragment, oligonucleotide, and biological sample. In some cases, excess antibody or antibody fragment can be washed away with an appropriate wash buffer after an incubation period sufficient to allow binding between the antibody or antibody fragment and the sample. Following this, the first oligonucleotide can be contacted with a second oligonucleotide, such that the first binding region of the second oligonucleotide can pair with the first oligonucleotide. The second oligonucleotide can be in a volume of a suitable buffer which can be capable of maintaining the integrity of all present elements. In some cases, excess second oligonucleotide can be washed away with an appropriate wash buffer after an incubation period sufficient to allow binding between the first oligonucleotide and second oligonucleotide. Following this, the second oligonucleotide can be contacted with a third oligonucleotide comprising a detection component. The third oligonucleotide can pair with the second binding region of the second oligonucleotide. The third oligonucleotide can be in a volume of a suitable buffer which can be capable of maintaining the integrity of all present elements. Excess third oligonucleotide can be washed away with an appropriate wash buffer after an incubation period sufficient to allow binding between the second oligonucleotide and third oligonucleotide. In this manner, an element of the biological sample can be connected to the antibody or antibody fragment, connected to the first oligonucleotide, connected to the second oligonucleotide, connected to the third oligonucleotide, connected to the detection element. Thus, the detection of the detection element may be performed to detect the element of the biological sample.

### Example 2: Longer Chains

In some cases, a method may resemble that in example 1, with the exception of the second oligonucleotide. In such methods, an additional 1, 2, 3, 4, 5, or more oligonucleotides can be incubated with the sample after the second oligonucleotide and before the third oligonucleotide, forming a chain of oligonucleotides which can make up the second oligonucleotide. In some cases, this may be pre-performed, such that the second oligonucleotide can comprise multiple oligonucleotides as a chain of oligonucleotides prior to contacting the first oligonucleotide.

### Example 3: 3-Dimensional Element Detection

In some embodiments, a sample may be a 3-dimensional sample, such as a frozen block of tissue. In such embodiments, a 3-dimensional detection of elements can be accomplished. This can be accomplished by several methods, which may include two types of methods described herein.

In the first type of methods, the block of tissue can be sliced with a microtome from the top to the bottom. After slicing the first slice, a method comprising contacting the biological sample, directly of the surface of the block of sample, with an antibody fragment that is conjugated to a first oligonucleotide, followed by contacting the first oligonucleotide with a first binding region of a second oligonucleotide, followed by contacting a second binding region of the second oligonucleotide with a third oligonucleotide, wherein the third oligonucleotide comprises a detection component, thereby connectively coupling the biological sample to the detection component. In some cases, the detection component can be detected, for example using a camera. Following detection, a new slice can be sliced, and the method can be repeated. The method can be considered complete upon performing these steps in at least 10%, at least 20%, at least 30%, at least 40%, at least 50%, at least 60%, at least 70%, at least 80%, at least 90%, or at least 100% of the sample. Upon completion of this method, the data from each slice can be combined in a manner which produces a 3-dimensional data set, which can indicate the locations of detected elements in the sample in three dimensions.

In the second type of methods, the block of tissue can be sliced with a microtome from the top to the bottom, such that the slices may be transferred to a slide. The slice on the slide can then be subjected to a method comprising contacting the biological sample with an antibody fragment that is conjugated to a first oligonucleotide, followed by contacting the first oligonucleotide with a first binding region of a second oligonucleotide, followed by contacting a second binding region of the second oligonucleotide with a third oligonucleotide, wherein the third oligonucleotide comprises a detection component, thereby connectively coupling the biological sample to the detection component. In some cases, the detection component can be detected, for example using a camera. Following detection, the method can be repeated for other slides containing slices from the same block of tissue. The method can be considered complete upon performing these steps in at least 10%, at least 20%, at least 30%, at least 40%, at least 50%, at least 60%, at least 70%, at least 80%, at least 90%, or at least 100% of the sample. Upon completion of this method, the data from each slide can be combined in a manner which produces a 3-dimensional data set, which can indicate the locations of detected elements in the sample in three dimensions.

In either type of methods and additional similar types of methods, slices can be about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 110 μm, about 120 μm, about 130 μm, about 140 μm, about 150 μm, about 160 μm, about 170 μm, about 180 μm, about 190 μm, about 200 μm, about 150 μm, about 300 μm, about 350 μm, about 400 μm, about 450 μm, about 500 μm, about 600 μm, about 700 μm, about 800 μm, about 900 μm, or about 1000 μm thick. In some cases, slices can be skipped, as in some slices can be removed without performing the method. Skipping slices can occur if the slices do not intersect a region of interest, due to time constraints, if a lower resolution is acceptable, or for other reasons. If slices are skipped, in some cases at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 slices may be skipped. In some cases, slices may be skipped uniformly throughout the sample. In some cases, certain parts of a sample may have more skipped slices than other parts. In some cases, no slices may be skipped. In some cases, the decision of whether or not to skip a slice may be made after analysis of the previous slice.

### Example 4: Element Detection Followed by Laser Capture Microdissection

In some cases, methods herein may be performed such that laser capture microdissection (LCM) can be performed in conjunction with the method. In such cases, the methods may be performed on specialty slides designed for LCM. In some cases, regions of a sample with no signal may be captured using LCM. In some cases, regions of a sample with signal may be captured using LCM. In some cases, regions of a sample with signal above a threshold may be captured using LCM. In some cases, regions of a sample with a signal below a threshold may be captured using LCM. In some cases, regions of a sample with a signal above a first threshold and below a second threshold may be captured using LCM.

Tissue captured using LCM can be further analyzed. In some cases, this further analysis can comprise chromatographic analysis, for example HPLC, GCMS, LCMS, or other chromatographic methods, western blotting, genotyping, PCR analysis, or other analysis technique.

### Example 5: Element Detection Using HRP

In some cases, methods herein may be performed using HRP as the detection component. In such cases, the method can be performed as described herein. Upon reaching the detection step, HRP can be exposed to an organic substrate and hydrogen peroxide. In some cases, the substrate can be luminol. If the substrate is luminol, then luminescence can be detected. If the substrate is ABTS, OPD, AmplexRed, Homovanillic acid, TMB, AEC, DAB, then the detection can be colorimetric.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method comprising:
contacting a biological sample with an antibody or antibody fragment that is conjugated to a first oligonucleotide;
contacting said first oligonucleotide with a first binding region of a second oligonucleotide;
contacting a second binding region of said second oligonucleotide with a third oligonucleotide, wherein said third oligonucleotide comprises a detection component;
thereby connectively coupling said biological sample to said detection component.

2. The method of claim 1, wherein said biological sample comprises at least one component selected from the group consisting of: cultured cells, biological tissue, biological fluid, a homogenate, and an unknown biological sample.

3. The method of claim 1, wherein said biological sample comprises material that is selected from the group consisting of: human origin, mouse origin, rat origin, cow origin, pig origin, sheep origin, rabbit origin, monkey origin, fruit fly origin, frog origin, nematode origin, fish origin, hamster origin, guinea pig origin, and woodchuck origin.

4. The method of claim 1, wherein said biological sample comprises material that is selected from the group consisting of: animal origin, plant origin, bacteria origin, fungus origin, and protist origin.

5. The method of claim 1, wherein said biological sample comprises a component selected from the group consisting of: virus, viral vector, and prion.

6. The method of claim 1, wherein said biological sample is fresh, frozen, or fixed.

7. The method of claim 1, wherein said biological sample is immobilized on a surface.

8. The method of claim 1, wherein said surface is a slide, a plate, a well, a tube, a membrane, a film, or a bead.

9. The method of claim 1, wherein said biological sample is immobilized within a three-dimensional structure.

10. The method of claim 9, wherein said three-dimensional structure is a frozen tissue, a paraffin block, or a frozen liquid.

11. The method of claim 1, wherein said antibody or antibody fragment comprises an IgG, an IgM, a monoclonal antibody, a scFv, a nanobody, a Fab, or a diabody.

12. The method of claim 1, wherein said antibody or antibody fragment is specific for an element of the sample.

13. The method of claim 12, wherein said element of the sample is frozen-fixed sample, a protein, a DNA molecule, an RNA molecule, or a lipid.

14. The method of claim 1, wherein said first oligonucleotide comprises a plurality of ribonucleic acids.

15. The method of claim 1, wherein said first oligonucleotide comprises a plurality of deoxyribonucleic acids.

16. The method of claim 1, wherein said first oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

17. The method of claim 1, wherein said first oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

18. The method of claim 1, wherein said first oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

19. The method of claim 1, wherein said first oligonucleotide comprises one or more synthetic nucleotides.

20. The method of claim 1, wherein said first oligonucleotide is wholly single stranded.

21. The method of claim 1, wherein said first oligonucleotide is partially double stranded.

22. The method of claim 1, wherein said first binding region of said second oligonucleotide is complimentary to at least a portion of said first oligonucleotide.

23. The method of claim 1, wherein said first binding region of the second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

24. The method of claim 1, wherein said first binding region of the second oligonucleotide is between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

25. The method of claim 1, wherein said first binding region of the second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

26. The method of claim 1, wherein said first binding region of said second oligonucleotide comprises one or more synthetic nucleotides.

27. The method of claim 1, wherein said second oligonucleotide comprises a plurality of ribonucleic acids.

28. The method of claim 1, wherein said second oligonucleotide comprises a plurality of deoxyribonucleic acids.

29. The method of claim 1, wherein said second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

30. The method of claim 1, wherein said second oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

31. The method of claim 1, wherein said second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

32. The method of claim 1, wherein said second oligonucleotide comprises one or more synthetic nucleotides.

33. The method of claim 1, wherein said second oligonucleotide is wholly single stranded.

34. The method of claim 1, wherein said second oligonucleotide is partially double stranded.

35. The method of claim 1, wherein said second binding region of said second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

36. The method of claim 1, wherein said second binding region of said second oligonucleotide is between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

37. The method of claim 1, wherein said second binding region of said second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

38. The method of claim 1, wherein said second binding region of said second oligonucleotide comprises one or more synthetic nucleotides.

39. The method of claim 1, wherein said second binding region of said second oligonucleotide is complimentary to at least a portion of said third oligonucleotide.

40. The method of claim 1, wherein said third oligonucleotide comprises a plurality of ribonucleic acids.

41. The method of claim 1, wherein said third oligonucleotide comprises a plurality of deoxyribonucleic acids.

42. The method of claim 1, wherein said third oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

43. The method of claim 1, wherein said third oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

44. The method of claim 1, wherein said third oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

45. The method of claim 1, wherein said third binding region of said second oligonucleotide comprises one or more synthetic nucleotides.

46. The method of claim 1, wherein said third oligonucleotide is wholly single stranded.

47. The method of claim 1, wherein said third oligonucleotide is partially double stranded.

48. The method of claim 1, wherein said third oligonucleotide is partially complimentary to said second binding region of said second oligonucleotide.

49. The method of claim 1, wherein said third oligonucleotide is fully complimentary to said second binding region of said second oligonucleotide.

50. The method of claim 1, wherein said detection component comprises a fluorophore, a radioactive isotope, or a compound capable of producing a colorimetric reaction.

51. The method of claim 1, wherein said detection component is located at the 3′ end of said third oligonucleotide.

52. The method of claim 1, wherein said detection component is located at the 5′ end of said third oligonucleotide.

53. The method of claim 1, wherein said detection component is located between the 3′ end and the 5′ end of said third oligonucleotide.

54. The method of claim 1, wherein said detection component be removed.

55. The method of claim 1, further comprising the step of immobilizing said biological sample on a surface prior to contacting said sample with said antibody or antibody fragment.

56. The method of claim 1, further comprising detection of the detection component after contacting said second binding region of said second oligonucleotide with said third oligonucleotide.

57. The method of claim 1, wherein the method is performed in a stepwise fashion.

58. The method of claim 1, wherein one or more steps are performed simultaneously.

59. The method of claim 1, wherein laser capture microdissection is performed after contacting said second binding region of said second oligonucleotide with said third oligonucleotide.

60. A kit comprising:
an antibody or antibody fragment that is conjugated to a first oligonucleotide;
a second oligonucleotide comprising a first binding region and a second binding region, wherein the first binding region of said second oligonucleotide is complimentary to at least a portion of said first oligonucleotide; and
a third oligonucleotide comprising a detection component, wherein said second binding region of said second oligonucleotide is complimentary to at least a portion of said third oligonucleotide.

61. The kit of claim 60, wherein said antibody or antibody fragment comprises an IgG, an IgM, a monoclonal antibody, a scFv, a nanobody, a Fab, or a diabody.

62. The kit of claim 60, wherein a non-specific bound antibody comprises less than 1%, less than 2%, less than 3%, less than 4%, less than 5%, less than 10%, less than 15%, less than 20%, less than 25%, less than 30%, less than 35%, or less than 40% of said antibody bound to the sample.

63. The kit of claim 60, wherein said first oligonucleotide comprises a plurality of ribonucleic acids.

64. The kit of claim 60, wherein said first oligonucleotide comprises a plurality of deoxyribonucleic acids.

65. The kit of claim 60, wherein said first oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

66. The kit of claim 60, wherein said first oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

67. The kit of claim 60, wherein said first oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

68. The kit of claim 60, wherein said first oligonucleotide comprises one or more synthetic nucleotides.

69. The kit of claim 60, wherein said first oligonucleotide is wholly single stranded.

70. The kit of claim 60, wherein said first oligonucleotide is partially double stranded.

71. The kit of claim 60, wherein said first binding region of said second oligonucleotide is complimentary to at least a portion of said first oligonucleotide.

72. The kit of claim 60, wherein said first binding region of the second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

73. The kit of claim 60, wherein said first binding region of the second oligonucleotide is between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

74. The kit of claim 60, wherein said first binding region of the second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

75. The kit of claim 60, wherein said first binding region of said second oligonucleotide comprises one or more synthetic nucleotides.

76. The kit of claim 60, wherein said second oligonucleotide comprises a plurality of ribonucleic acids.

77. The kit of claim 60, wherein said second oligonucleotide comprises a plurality of deoxyribonucleic acids.

78. The kit of claim 60, wherein said second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

79. The kit of claim 60, wherein said second oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

80. The kit of claim 60, wherein said second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

81. The kit of claim 60, wherein said second oligonucleotide comprises one or more synthetic nucleotides.

82. The kit of claim 60, wherein said second oligonucleotide is wholly single stranded.

83. The kit of claim 60, wherein said second oligonucleotide is partially double stranded.

84. The kit of claim 60, wherein said second binding region of said second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

85. The kit of claim 60, wherein said second binding region of said second oligonucleotide is between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

86. The kit of claim 60, wherein said second binding region of said second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

87. The kit of claim 60, wherein said second binding region of said second oligonucleotide comprises one or more synthetic nucleotides.

88. The kit of claim 60, wherein said second binding region of said second oligonucleotide is complimentary to at least a portion of said third oligonucleotide.

89. The kit of claim 60, wherein said third oligonucleotide comprises a plurality of ribonucleic acids.

90. The kit of claim 60, wherein said third oligonucleotide comprises a plurality of deoxyribonucleic acids.

91. The kit of claim 60, wherein said third oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

92. The kit of claim 60, wherein said third oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

93. The kit of claim 60, wherein said third oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

94. The kit of claim 60, wherein said third binding region of said second oligonucleotide comprises one or more synthetic nucleotides.

95. The kit of claim 60, wherein said third oligonucleotide is wholly single stranded.

96. The kit of claim 60, wherein said third oligonucleotide is partially double stranded.

97. The kit of claim 60, wherein said third oligonucleotide is partially complimentary to said second binding region of said second oligonucleotide.

98. The kit of claim 60, wherein said third oligonucleotide is fully complimentary to said second binding region of said second oligonucleotide.

99. The kit of claim 60, wherein said detection component comprises a fluorophore, a radioactive isotope, or a compound capable of producing a colorimetric reaction.

100. The kit of claim 60, wherein said detection component is located at the 3′ end of said third oligonucleotide.

101. The kit of claim 60, wherein said detection component is located at the 5′ end of said third oligonucleotide.

102. The kit of claim 60, wherein said detection component is located between the 3′ end and the 5′ end of said third oligonucleotide.

103. The kit of claim 60, wherein said detection component can be removed.

104. A composition comprising:
an antibody or antibody fragment that is conjugated to a first oligonucleotide;
wherein said first oligonucleotide is connected via base pairs to a first binding region of a second oligonucleotide;
wherein a second binding region of said second oligonucleotide is connected via base pairs to a third oligonucleotide; and
wherein said third oligonucleotide comprises a detection component.

105. The composition of claim 104, wherein said antibody or antibody fragment comprises an IgG, an IgM, a monoclonal antibody, a scFv, a nanobody, a Fab, or a diabody.

106. The composition of claim 104, wherein a non-specific bound antibody comprises less than 1%, less than 2%, less than 3%, less than 4%, less than 5%, less than 10%, less than 15%, less than 20%, less than 25%, less than 30%, less than 35%, or less than 40% of said antibody bound to the sample.

107. The composition of claim 104, wherein said first oligonucleotide comprises a plurality of ribonucleic acids.

108. The composition of claim 104, wherein said first oligonucleotide comprises a plurality of deoxyribonucleic acids.

109. The composition of claim 104, wherein said first oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

110. The composition of claim 104, wherein said first oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

111. The composition of claim 104, wherein said first oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

112. The composition of claim 104, wherein said first oligonucleotide comprises one or more synthetic nucleotides.

113. The composition of claim 104, wherein said first oligonucleotide is wholly single stranded.

114. The composition of claim 104, wherein said first oligonucleotide is partially double stranded.

115. The composition of claim 104, wherein said first binding region of said second oligonucleotide is complimentary to at least a portion of said first oligonucleotide.

116. The composition of claim 104, wherein said first binding region of the second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

117. The composition of claim 104, wherein said first binding region of the second oligonucleotide is between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

118. The composition of claim 104, wherein said first binding region of the second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

119. The composition of claim 104, wherein said first binding region of said second oligonucleotide comprises one or more synthetic nucleotides.

120. The composition of claim 104, wherein said second oligonucleotide comprises a plurality of ribonucleic acids.

121. The composition of claim 104, wherein said second oligonucleotide comprises a plurality of deoxyribonucleic acids.

122. The composition of claim 104, wherein said second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

123. The composition of claim 104, wherein said second oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

124. The composition of claim 104, wherein said second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

125. The composition of claim 104, wherein said second oligonucleotide comprises one or more synthetic nucleotides.

126. The composition of claim 104, wherein said second oligonucleotide is wholly single stranded.

127. The composition of claim 104, wherein said second oligonucleotide is partially double stranded.

128. The composition of claim 104, wherein said second binding region of said second oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

129. The composition of claim 104, wherein said second binding region of said second oligonucleotide is between 5-10, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

130. The composition of claim 104, wherein said second binding region of said second oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

131. The composition of claim 104, wherein said second binding region of said second oligonucleotide comprises one or more synthetic nucleotides.
The method of claim 1, wherein said second binding region of said second oligonucleotide is complimentary to at least a portion of said third oligonucleotide.

132. The composition of claim 104, wherein said third oligonucleotide comprises a plurality of ribonucleic acids.

133. The composition of claim 104, wherein said third oligonucleotide comprises a plurality of deoxyribonucleic acids.

134. The composition of claim 104, wherein said third oligonucleotide is at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long.

135. The composition of claim 104, wherein said third oligonucleotide is between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length.

136. The composition of claim 104, wherein said third oligonucleotide is no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

137. The composition of claim 104, wherein said third binding region of said second oligonucleotide comprises one or more synthetic nucleotides.

138. The composition of claim 104, wherein said third oligonucleotide is wholly single stranded.

139. The composition of claim 104, wherein said third oligonucleotide is partially double stranded.

140. The composition of claim 104, wherein said third oligonucleotide is partially complimentary to said second binding region of said second oligonucleotide.

141. The composition of claim 104, wherein said third oligonucleotide is fully complimentary to said second binding region of said second oligonucleotide.

142. The composition of claim 104, wherein said detection component comprises a fluorophore, a radioactive isotope, or a compound capable of producing a colorimetric reaction.

143. The composition of claim 104, wherein said detection component is located at the 3′ end of said third oligonucleotide.

144. The composition of claim 104, wherein said detection component is located at the 5′ end of said third oligonucleotide.

145. The composition of claim 104, wherein said detection component is located between the 3′ end and the 5′ end of said third oligonucleotide.

146. The composition of claim 104, wherein said detection component be removed.
